SEQUENCE LISTING (11 sequences)

SEQ ID NO: 1 - protein, 211 aa, Homo sapiens [sequence omitted]

SEQ ID NO: 2 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 3 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 4 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 5 - protein, 210 aa, Homo sapiens
MGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 6 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 7 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 8 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 9 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 10 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 11 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

(19) United States
(12) Patent Application Publication
(10) Pub. No.: US 2026/0199401 A1
(43) Pub. Date: Jul. 16, 2026

(54) USE OF CONDITIONED MEDIUM OF CORD LINING MESENCHYMAL STEM CELLS FOR HEALING AND/OR PREVENTING THE FORMATION AND/OR RECURRENCE OF A WOUND OR A DAMAGED SKIN AREA

(71) Applicant: CELLRESEARCH CORPORATION PTE. LTD., Singapore (SG)

(72) Inventor: Toan Thang PHAN, Singapore (SG)

(73) Assignee: CELLRESEARCH CORPORATION PTE. LTD., Singapore (SG)

(21) Appl. No.: 19/135,038

(22) PCT Filed: Dec. 9, 2022
(86) PCT No.: PCT/SG2022/050897

§ 371 Date: Jun. 2, 2025

(51) Int. Cl. A61K 35/51; A61K 9/00; A61K 31/12; A61K 35/28; A61K 36/28; A61P 17/02; C12N 5/0775

(52) U.S. Cl. CPC A61K 35/51; A61K 9/0014; A61K 31/12; A61K 35/28; A61K 36/28; A61P 17/02; C12N 5/0665; C12N 2500/90; C12N 2501/11; C12N 2501/33

(57) ABSTRACT

The invention also refers to a method of inducing, stimulating and/or promoting healing of a wound or a damaged skin area, wherein the method comprises topically treating the skin of a subject surrounding the wound or damaged skin area with conditioned medium derived from cultivation of mesenchymal stem cells of the umbilical cord. Equally, the invention relates to a method of preventing formation and/or recurrence of a wound or a damaged skin area in a subject being at risk of developing a wound or a damaged skin area, wherein the method comprises topically treating the skin of the subject with conditioned medium derived from cultivation of mesenchymal stem cells of the umbilical cord. Further, the invention relates to a method of treating rosacea, psoriasis, eczema, dermatitis, topical steroid withdrawal syndrome, epidermolysis bullosa, or skin damage caused by fragile skin, wherein the method comprises topically treating the skin of a subject surrounding the wound or damaged skin area to be treated with conditioned medium derived from cultivation of mesenchymal stem cells of the umbilical cord. The present invention is also directed to conditioned medium, uses thereof, compositions comprising the same, methods of producing the same as well as uses of these compositions.

### FIELD OF THE INVENTION

The invention refers to a method of inducing, stimulating and/or promoting healing of a wound or a damaged skin area, wherein the method comprises topically administering conditioned medium derived from cultivation of mesenchymal stem cells of the umbilical cord to the skin of a subject surrounding the wound or damaged skin area with. The invention also relates to a method of preventing formation and/or recurrence of a wound or a damaged skin area in a subject being at risk of developing a wound or a damaged skin area, wherein the method comprises topically administering conditioned medium derived from cultivation of mesenchymal stem cells of the umbilical cord to the skin of the subject. Further, the invention relates to a method of treating rosacea, psoriasis, eczema, dermatitis, topical steroid withdrawal syndrome, epidermolysis bullosa, or skin damage caused by fragile skin, wherein the method comprises topically administering to the skin conditioned medium derived from cultivation of mesenchymal stem cells of the umbilical cord. The present invention is also directed to conditioned medium, uses thereof, as well as compositions comprising the same and methods of producing conditioned medium as well compositions containing conditioned medium. These methods and compositions can be therapeutic methods but also non-therapeutic or cosmetic methods or compositions.

### BACKGROUND OF THE INVENTION

Wounds and damaged skin and their healing complications as well as the lack to effectively prevent the development of the same by patients being at risk of developing a wound or damaged skin are a major problem both in hospital and home settings. The ability of an organism to activate the healing process of a wound or damaged skin effectively and promptly is not only essential for restoring the skin, which is continuously confronted with the external environment, but also because the skin serves as a primary defence system or protective barrier against the environment. Consequently, following an injury resulting in a wound or damaged skin, loss of the integrity of large portions of the skin may lead to major disability or even death due to an increased susceptibility to fungus, bacteria, and viral infections.

In order to understand the process of healing of damaged skin and wounds, i.e., the repair and regeneration of the skin, an understanding of the anatomic and physiologic functions of normal skin is essential. Histologically, the skin is divided into two functional layers, the epidermis and dermis, which are separated by a basement membrane. The epidermis is multicellular and new cells are continuously made in the deepest layer of the epidermis, the stratum basalis and move outward toward the surface of the skin. The dermis is responsible for the mechanical properties of the skin and is composed of fibers and connective tissue wherein the fibers are composed of elastin and collagen. Collagen fibers provide strength to the skin, while elastin fibers provide recoil strength. In normal skin, the dermis and epidermis form a protective barrier against the external environment and subserve a myriad of protective functions, including mechanical and photoprotection, immunosurveillance, nutrient metabolism and repair. Because the skin serves such an important role in the body, wounded skin needs to be replaced quickly. The physiological process of wound healing is initiated when the protective barrier of the dermis and epidermis is broken, the structural integrity is compromised and the underlying vasculature is disrupted.

Healing of wounds involves the processes of hemostasis, inflammation and proliferation. Skin repair begins with the clot formation and and platelet degranulation, which initiate homeostasis by releasing growth factors necessary for wound repair and promoting regeneration of injured epidermis. Early in the inflammatory phase, neutrophils are at the wound site and are primarily responsible for scavenging bacterial and foreign material. Late in the inflammatory phase, monocytes migrate to the wound site and transform into macrophages. The subsequent proliferative phase is characterized by the increased formation of granulation tissue where fibroblasts lay a bed of collagen and produce new capillaries, finally leading to the formation of a new, intact epidermis of the affected area.

However, there are many types of wounds and diseases and conditions associated with damaged skin that have different complications in which the formation and/or recurrence of a wound or damaged skin cannot be effectively be prevented and/or wherein the healing process is deficient or even inhibited. An illustrative example, the development of pressure ulcers also known as pressure sores or pressure bedsores is generally difficult to prevent. Pressure ulcers are injuries to the skin and underlying tissue, primarily caused by prolonged pressure to the skin and may develop gradually or formed within hours, thereby affecting any part of the body that is put under pressure, for example, pressure ulcers may particularly occur on bony parts of the body, such as the heels, elbows, hips and base of the spine. Typically, pressure ulcers affect, for example, patients confined to bed, who sit in a chair or wheelchair for long periods of time, older subjects or patients having mobility problems, subjects with skin that is easily damaged through dehydration, obese subjects or patients, subjects or patients having a medical condition that affect blood supply and makes the skin more fragile or cause movement problems such as but not limited to diabetes, peripheral arterial disease, kidney failure, heart failure, multiple sclerosis (MS) and Parkinson's disease. Countermeasures in order to reduce the risk of developing pressure ulcers may include regular repositioning of the body and monitoring skin properties for signs and symptoms of pressure ulcer. Thus, effective and reliable compositions and methods are still needed in order to prevent the formation and/or recurrence of a wound or damaged skin such as those caused by pressure ulcers.

Other healing complicated problems are known for chronic wounds. Chronic wounds are often associated with severe complications as they comprise increased levels of inflammatory cells and proteases which inhibit the healing process by degrading the extracellular matrix (ECM) which is comprised largely of collagen which plays a significant role in the healing response, particularly in the proliferation phase as outlined above. Examples for chronic wounds are diabetic chronic wounds, such as diabetic foot ulcers, which are caused by poor blood flow and inflammation, and are slow to heal, or may never heal if left untreated. This can lead to severe infections, among other problems. In such a case, natural wound healing of chronic diabetic wounds is difficult because elevated levels of pro-inflammatory cytokines are secreted which hamper natural wound healing requiring anti-inflammatory signals and epithelial and endothelial migration activities are hindered which are however essential for the process of closing the wound.

Typical procedures for treating wounds, particularly chronic wounds such as diabetic ulcers, venous ulcers, and pressure sores, include the use of dressings that are applied onto the wounds.

One example of an approved medical dressing is Apligraf, a living, bi-layered skin substitute, which contains Type I bovine collagen, extracted and purified from bovine tendons and viable allogeneic human fibroblast and keratinocyte cells isolated from human infant foreskin. This wound dressing/covering is, inter alia, indicated for use with standard diabetic foot ulcer care for treatment of full-thickness neuropathic diabetic foot ulcers of greater than 3 weeks duration which have not adequately responded to conventional ulcer therapy and which extend through the dermis but without tendon, muscle, capsule or bone exposure. A further wound dressing/covering is Dermagraft®, a living, bi-layered skin substitute, which contains Type I bovine collagen, extracted and purified from bovine tendons and viable allogeneic human fibroblast and keratinocyte cells isolated from human infant foreskin. Dermagraft® is indicated for use in the treatment of full-thickness diabetic foot ulcers greater than 6 weeks duration which extend through the dermis, but without tendon muscle, joint capsule or bone exposure. Dermagraft® should be used in conjunction with standard wound care regimens and in patients that have adequate blood supply to the involved foot.

However, these wound coverings are rather complex to manufacture and thus relatively expensive, thereby adding to the rather significant costs for treatment/management of diabetic foot ulcer. Thus, there is a need for new compositions that are easy to manufacture and yet are effective in treating ulcers such as diabetic foot ulcer. Accordingly, it is an object of the invention to provide such compositions for their subsequent use in wound healing.

### SUMMARY OF THE INVENTION

This object is accomplished by the methods, the conditioned medium and uses thereof having the features of the independent claims.

In a first aspect, the present invention provides a method of inducing, stimulating and/or promoting healing of a wound or a damaged skin area, wherein the method comprises topically administering conditioned medium derived from cultivation of mesenchymal stem cells of the umbilical cord to the skin of a subject surrounding the wound or damaged skin area.

In a second aspect, the present invention provides a method of preventing formation and/or recurrence of a wound or a damaged skin area in a subject being at risk of developing a wound or a damaged skin area, wherein the method comprises topically administering conditioned medium derived from cultivation of mesenchymal stem cells of the umbilical cord to the skin of a subject.

In a third aspect, the present invention provides a method of treating rosacea, psoriasis, eczema, dermatitis, topical steroid withdrawal syndrome, epidermolysis bullosa, or skin damage caused by fragile skin, wherein the method comprises topically treating the skin of a subject surrounding the wound or damaged skin area to be treated with conditioned medium derived from cultivation of mesenchymal stem cells of the umbilical cord.

In a fourth aspect, the present invention provides a method of producing a conditioned medium, the method comprising: (a) cultivating mesenchymal stem cells of the umbilical cord in a culture medium comprising DMEM (Dulbecco's modified eagle medium), F12 (Ham's F12 Medium), M171 (Medium 171) and FBS (Fetal Bovine Serum); (b) removing the mesenchymal stem cells of the umbilical cord from the culture medium; (c) optionally cultivating the mesenchymal stem cells of the umbilical cord in a further culture medium, optionally wherein the further cell culture medium comprises a water-soluble antioxidant, wherein the conditioned medium is obtained by collecting the cell culture medium.

In a fifth aspect, the present invention provides a conditioned medium derived from mesenchymal stem cells of the umbilical cord obtained or obtainable by the method according to the method of the present invention.

In a sixth aspect, the present invention provides a composition comprising the conditioned medium of the present invention. Such a composition can be a pharmaceutical or cosmetic composition, for example, in form of an ointment, lotion, cream or gel that can used for treating the wounds or skin conditions described herein.

In an eight aspect, the present invention provides the use of conditioned medium derived from cultivation of mesenchymal stem cells of the umbilical cord for the manufacture of a pharmaceutical composition for inducing, stimulating and/or promoting healing of a wound or damaged skin area and/or for preventing formation and/or recurrence of a wound or damaged skin area, and/or for treating rosacea, psoriasis, eczema, dermatitis, topical steroid withdrawal syndrome, epidermolysis bullosa.

In an ninth aspect, the present invention provides the use of conditioned medium derived from cultivation of mesenchymal stem cells of the umbilical cord for for inducing, stimulating and/or promoting healing of a wound or damaged skin area and/or for preventing formation and/or recurrence of a wound or damaged skin area, and/or for treating rosacea, psoriasis, eczema, dermatitis, topical steroid withdrawal syndrome, epidermolysis bullosa.

### BRIEF DESCRIPTION OF THE DRAWINGS

The invention will be better understood with reference to the detailed description when considered in conjunction with the non-limiting examples and the drawings, in which

FIG. 1 shows a technical information sheet of Lonza for Dulbecco's modified eagle medium, including the catalogue number of the DMEM used for the making of the illustrative example of a medium (PTT-6) in the Experimental Section.

FIG. 2 shows a technical information sheet of Lonza for Ham's F12 medium.

FIG. 3 shows a technical information sheet of Lonza for DMEM:F12 (1:1) medium, including the catalogue number of the DMEM:F12 (1:1) medium used for the making of the illustrative example of a medium (PTT-6) in the Experimental Section.

FIG. 4 shows a technical information sheet of Life Technologies Corporation for M171 medium, including the catalogue number of the M171 medium used for the making of the illustrative example of a medium (PTT-6) in the Experimental Section.

FIG. 5 shows a list of ingredients, including their commercial supplier and the catalogue number that have been used in the Experimental Section for making the medium PTT-6.

FIG. 6 shows the reduction in wound size (cm²) over time (week 1 to week 16) in 10 patients suffering from chronic diabetic foot ulcer (DFU) and treated with conditioned medium of the invention.

FIG. 7A-B shows image sequences of reduction in wound size (cm²) and healing of the wound in two patients suffering from chronic diabetic foot ulcer (DFU) over a total duration of 16 weeks (lower image untreated and upper images treated) and with two applications of conditioned medium of the invention per week.

FIG. 8 shows an image sequence of reduction in wound size (cm²) and healing of the wound in a patient suffering from chronic diabetic foot ulcer (DFU) over a total duration of 8 weeks (lower image untreated and upper images treated) and with three applications of conditioned medium of the invention per week.

FIG. 9A-B shows reduction in wound size (cm²) and healing of the wound in patients suffering from chronic diabetic foot ulcer (DFU) (left-untreated, right-treated) over a total duration of 5 weeks and with two applications of conditioned medium of the invention per week.

FIG. 10 shows reduction in wound size (cm²) and healing of the wound in a patient suffering from chronic diabetic foot ulcer (DFU) (left-untreated, right-treated) over a total duration of 3 weeks and with three applications of conditioned medium of the invention per week.

### DETAILED DESCRIPTION

The present invention is directed to methods and compositions which are all suitable for inducing, stimulating and/or promoting healing a wound or damaged skin area. Additionally, the methods and compositions of the present invention are all suitable for preventing formation and/or recurrence of a wound or damaged skin area in a subject being at risk of developing a wound or damaged skin area. Furthermore, the methods and compositions of the present invention are all suitable for treating rosacea, psoriasis, eczema, dermatitis, topical steroid withdrawal syndrome, epidermolysis bullosa, or skin damage caused by fragile skin.

It has been surprisingly found herein that conditioned medium derived from the cultivation of mesenchymal stem cells of the umbilical cord, particularly derived from the cultivation of mesenchymal stem cells of the amniotic membrane of the umbilical cord, has the effect of inducing, stimulating and/or promoting healing of a wound or skin damage without applying the conditioned medium directly into or onto a wound or damaged skin area itself but by administering the conditioned medium only to the skin surrounding the wound or damaged skin area, i.e. to a skin area which is not wounded or damaged. It has been particularly surprising that by this route of administration, the conditioned medium as described herein is even able to heal/close difficult to heal wounds such as chronic diabetic foot ulcers. See Example 3, which shows that 12 of 21 patients suffering from chronic diabetic foot ulcers (DFU) demonstrated total DFU closure. This finding of the invention allows to formulate the conditioned medium into a formulation such as a cream, a gel, or a lotion that can be easily manufactured (at a much lower costs than, for example, wound coverings such as Apligraf or Dermagraft), that is stable at room temperature, that can therefore be easily distributed to hospitals and treatment centers and that additionally can easily be applied, even by non-medically trained personnel or even the patients themselves. These advantages allow to provide treatment to patients suffering from chronic diabetic foot ulcers who, for example, in less developed countries, have up to now not been able to afford a treatment with wound coverings such as Apligraf or Dermagraf. In addition, by being able to close such chronic wounds, the present invention provides patients with the opportunity to, for example, return to work, thereby improving also the socio-economic status of the affected patient. Finally, by providing an affordable treatment option of chronic wounds such as diabetic foot ulcers, the present invention provides the opportunity to reduce overall costs for treatment of diabetic foot ulcer, in particular in countries with high prevalence of diabetes. Without wishing to be bound by theory, it is believed that the induction, stimulation, promotion of healing and/or prevention of wounds or damaged skin is caused by the ability of the conditioned medium to contain a high concentration one or more biological factors such as exosomes, growth factors, proteins, peptides and cytokines which contribute to build up a robust extracellular matrix and support skin repair when administered to the skin surrounding the wound or damaged skin area or to the intact skin of a subject being at risk of developing a wound or damaged skin area. By this means, the conditioned medium of the present invention provides excellent properties for inducing, stimulating and/or promoting healing of a wound or damaged skin area and/or for preventing formation and/or recurrence of a wound or damaged skin area in a subject being at risk of developing a wound or damaged skin area and/or for treating rosacea, psoriasis, eczema, dermatitis, topical steroid withdrawal syndrome, epidermolysis bullosa, or skin damage caused by fragile skin.

As demonstrated in the experimental section, patients with chronic and refractory ulcers are treated with conditioned medium of the present invention wherein conditioned medium is only administered to the skin surrounding the wound and not onto the wound itself. Patients experience a significant improvement in wound reduction which is observed within 16, 8, 5 or even within 3 weeks. Particularly, promoting wound healing, e.g., through increased skin cell turnover, promoting the formation of extracellular matrix, facilitating regenerative healing through potent anti-inflammatory cytokines and promoting healing through angiogenesis can be attributed to the fact that the conditioned medium is particularly rich in growth factors and other biological factors positively implicated with the repair and regeneration of skin such as but not limited to PDGF-AA, PDGF-BB, transforming growth factors (TGFs), insulin-like growth factors (IGFs), fibroblast growth factors (FGFs), vascular endothelial growth factors (VEGFs), keratinocytes growth factors (KGFs), interleukin-10 (IL-10), angiopoietin 1 (Ang-1) and/or hepatocyte growth factor (HGF).

Conditioned medium of the invention may comprise at least one and, more typically, a combination of biological factors produced by mesenchymal stem cells of the umbilical cord (e.g., at least one and more typically, a combination of biological factors listed above). For example, they can comprise one or more (at least one) angiogenic cytokines and/or growth factors. For example, they can comprise one or more selected from the group consisting of PDGF-AA, PDGF-BB, transforming growth factors (TGFs), insulin-like growth factors (IGFs), fibroblast growth factors (FGFs), vascular endothelial growth factors (VEGFs), keratinocytes growth factors (KGFs) and interleukin-10 (IL-10), angiopoietin 1 (Ang-1) and/or hepatocyte growth factor (HGF).

In this regard, growth factors are generally known as being capable of stimulating cellular growth and/or proliferation and/or cellular differentiation and the positive involvement of growth factors on skin and particularly wound healing is known to the person skilled in the art. For example, for the involvement of Angiopoietin 1 in wound healing, see, for example, Li et al. Stem Cell Research & Therapy 2013, 4:113 “Mesenchymal stem cells modified with angiopoietin-1 gene promote wound healing” or Bitto et al, “Angiopoietin-1 gene transfer improves the impaired wound healing of the genetically diabetic mice without increasing VEGF expression”, Clinical Science May 14, 2008, 114 (12) 707-718. For the involvement of Hepatocyte Growth Factor (HGF) in wound healing, in particular healing of chronic/non-healing wounds, see for example, Yoshida et al., “Neutralization of Hepatocyte Growth Factor Leads to Retarded CutaneousWound Healing Associated with Decreased Neovascularization and Granulation Tissue Formation” J. Invest. Dermatol. 120:335-343, 2003, Li, Jin-Feng et al. “HGF Accelerates Wound Healing by Promoting the Dedifferentiation of Epidermal Cells through ß1-Integrin/ILK Pathway.” BioMed Research International 2013 (2013): 470418 or Conway et al, “Hepatocyte growth factor regulation: An integral part of why wounds become chronic”. Wound Rep Reg (2007) 15 683-692. For the involvement of Vascular Endothelial Growth Factor (VEGF) in wound healing, in particular healing of chronic/non-healing wounds, see for example Froget et al., Eur. Cytokine Netw., Vol. 14, March 2003, 60-64 or Bao et al., “The Role of Vascular Endothelial Growth Factor in Wound Healing” J Surg Res. 2009 May 15; 153 (2): 347-358. For the involvement of Transforming Growth Factor Beta (including TGF-ß1, TGF-ß2, and TGF-ß3) in wound healing, in particular healing of chronic/non-healing wounds see for example, Ramirez et al. “The Role of TGFb Signaling in Wound Epithelialization” Advances In Wound Care, Volume 3, Number 7, 2013, 482-491 or Pakyari et al., Critical Role of Transforming Growth Factor Beta in Different Phases of Wound Healing, Advances In Wound Care, Volume 2, Number 5, 2012, 215-224.

Further, the generally known positive implication of growth factors on skin and particularly wound healing is, e.g., shown by their ability to enhance elastin and hyaluronic acid expression in human skin fibroblasts (see Kähäri, V. M., et al., Transforming growth factor-beta up-regulates elastin gene expression in human skin fibroblasts. Evidence for post-transcriptional modulation. Lab Invest, 1992. 66(5): p. 580-8; and Takami, Y., et al., Modulation of hepatocyte growth factor induction in human skin fibroblasts by retinoic acid. Biochimica et Biophysica Acta (BBA)—Molecular Cell Research, 2005. 1743(1): p. 49-56). For example, mesenchymal stem cells of the umbilical cord can produce one or more factors which are involved in, for example, cell proliferation and migration (e.g., Vascular Endothelial Growth Factor (VEGF); Platelet-derived growth factor AA (PDGF-AA); Basic Fibroblast Growth Factor (bFGF) and Insulin-Like Growth Factor 1 (IGF1); promoting angiogenesis (e.g., Vascular Endothelial Growth Factor (VEGF); anti-inflammation (e.g.; Transforming Growth Factor Beta-1 (TGF-β1)); promoting elastin and/or hyaluronic acid expression (e.g., Transforming Growth Factor Beta-1 (TGF-31); Basic Fibroblast Growth Factor (bFGF); Insulin-Like Growth Factor 1 (IGF1)) as well as other factors.

The conditioned medium described here may contain exosomes. The skilled person knows that exosomes are positively implicated with wound healing processes, e.g., by conferring anti-inflammatory properties, promotion of angiogenesis and stimulation of new tissue formation; see Danyang Li, Na Wu, Mechanism and application of exosomes in the wound healing process in diabetes mellitus, Diabetes Research and Clinical Practice, Volume 187, 2022; Li, X., Xie, X., Lian, W. et al. Exosomes from adipose-derived stem cells overexpressing Nrf2 accelerate cutaneous wound healing by promoting vascularization in a diabetic foot ulcer rat model. Exp Mol Med 50, 1-14 (2018). In this regard, application of conditioned medium of the present invention has beneficial effects on all stages of wound healing, e.g., by promoting wound or damaged skin repair and/or preventing the formation and/or recurrence of a wound or damaged skin.

The finding of the present invention is that conditioned medium derived from cultivation of mesenchymal cells of the umbilical cord, wherein the cells inter alia secrete growth factors and other biological factors into the conditioned medium, induces, stimulates and/or promotes healing of a wound or damaged skin, prevents formation and/or recurrence of a wound or damaged skin area in a subject being at risk of developing a wound or damaged skin area and/or is for treating rosacea, psoriasis, eczema, dermatitis, topical steroid withdrawal syndrome, epidermolysis bullosa, or skin damage caused by fragile skin. By this means, conditioned medium of the present invention is believed to derive high concentration of growth factors and/or other biological factors, which can effectively aid in healing a wound or healing a damaged skin area and/or prevent formation and/or recurrence of such a wound or damaged skin area and/or for treating rosacea, psoriasis, eczema, dermatitis, topical steroid withdrawal syndrome, epidermolysis bullosa, or skin damage caused by fragile skin.

In this context, it is noted that cultivation of mesenchymal stem cells of the umbilical cord in the culture medium as described herein provides for the isolation of an extremely homogenous and well-defined mesenchymal stem cell population of umbilical cord tissue (for example, a mesenchymal stem cell population of the Wharton's Jelly or of the amniotic membrane of the umbilical cord) as disclosed in WO 2019/199234 A1 or WO 2018/067071 A1. Particularly, the isolation of a mesenchymal stem cell population of the umbilical cord results in more than 90%, or even 99% or more of the cells that are positive for the three mesenchymal stem cell markers CD73, CD90 and while at the same these stem cells lack expression of CD34, CD45 and HLA-DR (see, e.g., WO 2019/199234 A1 or WO2018/067071 A1), meaning that, e.g., 99% or even more cells of this population express the stem cell markers CD73, CD90 and CD105 while not expressing the markers CD34, CD45 and HLA-DR. In this context, such a mesenchymal stem cell population of the umbilical cord (tissue) fully meet the criteria generally accepted for human mesenchymal stem cells to be used for cellular therapy as defined, for example, by Dominici et al, “Minimal criteria for defining multipotent mesenchymal stromal cells. The International Society for Cellular Therapy position statement”, Cytotherapy (2006) Vol. 8, No. 4, 315-317, Sensebe et al., “Production of mesenchymal stromal/stem cells according to good manufacturing practices: a, review”, Stem Cell Research & Therapy 2013, 4:66), Vonk et al., Stem Cell Research & Therapy (2015) 6:94, or Kundrotas Acta Medica Lituanica. 2012. Vol. 19. No. 2. P. 75-79. Accordingly, this in turn means that conditioned medium which is derived from cultivation of such an extremely homogenous and well-defined cell population is ideal to derive highly homogenous and well-defined components, such as highly concentrated growth factors, in the conditioned medium. Moreover, the use of conditioned medium of the present invention in wound healing or healing a damaged skin area and/or preventing formation and/or recurrence of such a wound or damaged skin area has further several advantages over stem-cell based applications since donor-recipient matching problems by stem-cell based treatment or transplantation can be avoided and conditioned medium is easier to prepare and is cheaper compared to stem-cell based skin therapy.

The conditioned medium derived from cultivation of mesenchymal stem cells of the umbilical cord that is being rendered suitable in healing and/or treating a wound or damaged skin area as well as in preventing formation and/or recurrence of such a wound or damaged skin area may be derived from cultivation of any suitable mesenchymal stem cell population of the umbilical cord (tissue) known in the art. For example, the mesenchymal stem cells of the umbilical cord may be derived from any mammalian tissue or compartment/body part known to contain mesenchymal stem cells of the umbilical cord. In illustrative examples, the mesenchymal stem cells may be mesenchymal stem cells of the amniotic membrane of umbilical cord. The effects of promoting healing, treating and/or preventing of a wound or damaged skin are achieved, e.g., because these stem cells secrete growth factors and/or other biological factors positively implicated in healing a wound. For example, biological factors that may be in the conditioned medium include but are not limited to growth factors, nucleic acids such as mRNA or DNA, exosomes, hormones, cytokines, extracellular matrix, proteins, vesicles, antibodies, chemokines, receptors, inhibitor, and granules. Such a conditioned medium, and combinations of any of growth factors and/or biological factors comprised therein can be used in the method of the present invention.

The present invention relates, in a first aspect, to a method of inducing, stimulating and/or promoting healing of a wound or a damaged skin area, wherein the method comprises topically administering conditioned medium derived from cultivation of mesenchymal stem cells of the umbilical cord to the skin of a subject surrounding the wound or the damaged skin area. A wound or damaged skin as used herein and in the context of the present invention refers to an injury or damage to the integrity of skin, particularly the skin surface, and may also include injury or damage to underlying tissues. For example, the wound or damaged skin is caused by a burn, a bite, a trauma, a surgery or a disease such as a skin disease or condition or a metabolic disorder. The skin disease or skin condition or metabolic disorder can be any disease, conditioned or disorder associated with the occurrence of a wound or damaged skin, particularly in which healing of a wound or damaged skin is wanted/required. Non-limiting examples of such a skin disease, skin condition or a metabolic disorder are Type I or Type II diabetes with suffering from chronic ulcers such as diabetic foot ulcer (DFU), pressure ulcer, limb ischemia and venous leg ulcer, rosacea, psoriasis, eczema, dermatitis, topical steroid withdrawal syndrome, epidermolysis bullosa, or skin damage caused by fragile skin. In some embodiments, the wound or damaged skin is a chronic wound or a chronically damaged skin. In some embodiments, the damaged skin area is a wound. In some embodiments, the wound or damaged skin area is a wound, wherein the wound is selected from the group consisting of diabetic foot ulcer (DFU), pressure ulcer, a wound due to chronic limb ischemia, a wound due to chronic limb-threatening limb ischemia, venous leg ulcer, a surgical wound, a stab wound, a burn wound, or a bite wound.

By “inducing, stimulating and/or promoting healing of a wound or damaged skin” as used herein is meant the ability of the conditioned medium to increase and/or start (induce) healing process by repair and/or regeneration of the skin, particularly in patients suffering from a wound or a damaged skin area that is associated with complications of healing. The ability to increase and/or start wound healing can be attributed to the presence of growth factors and other biological factors such as exosomes. The secretion of growth factors and other biological factors into the culture medium (and thus the growth factors and biological factors contained in the conditioned medium of the present invention) can be measured/determined with any suitable method for example, by measuring the amount of growth factors and biological factors by means of commercially available antibodies/immunoassays (cf., the Experimental Section). Such measurements can be made in an automated fashion, using, for example a system such as the FLEXMAP 3D system (Luminex Corporation, Austin, Texas, USA).

The present invention also relates, in a second aspect, to a method of preventing formation and/or recurrence of a wound or a damaged skin area in a subject being at risk of developing a wound or a damaged skin area, wherein the method comprises topically treating the skin of a subject with conditioned medium derived from cultivation of mesenchymal stem cells of the umbilical cord. By “preventing the formation and/or recurrence of a wound or damaged skin” as used herein is meant the ability of the conditioned medium of the invention to avoid and/or reduce the risk of the occurrence of a wound or damaged skin in a subject which is at risk of developing a wound or skin damage wherein the risk may be due to a previous skin disease, skin condition or metabolic disease or the like. For preventing the formation and/or recurrence of a wound or damaged skin, the conditioned medium can be administered to the functionally and physiologically intact skin area of a subject.

The conditioned medium of the present invention used for treatment may, for example, be applied at a final amount of, for example, at about 5% or 10% to about 80% and more preferably at a final amount of about 10% to about 50%, more preferably of about 10% to about 30%, more preferably of about 20% to about 30%, more preferably of about 20%, wherein the % can either refer to % (v/v) of the total volume of the composition or to % (w/w) of the total weight of the composition that contains the conditioned medium. Cf., in this respect the illustrative examples of specific formulations described herein. For this purpose, the conditioned medium of the present invention can be diluted in (formulated with) any suitable diluent/carrier medium. The carrier medium may be a liquid, a gel or a cream-like formulation. For example, the conditioned medium may be diluted in a carrier medium such as PBS, water or a basal medium to name only a few suitable media. A basal medium as used herein refers to a mixture containing saccharides, amino acids, water, etc., required for the survival of a cell and include commercial prepared medium such as but not limited to Dulbecco's Modified Eagle's Medium (DMEM), Endothelial differentiation medium (EDM), Minimal Essential Medium (MEM), Basal Medium Eagle (BME), RPMI 1640, F-10, F-12, α-Minimal Essential Medium (α-MEM), Glasgow's Minimal Essential Medium (G-MEM), and Iscove's Modified Dulbecco's Medium. In some examples, the conditioned medium of the present invention may be comprised in a solution that comprises DMEM. This medium may further comprise an antioxidant such as a soluble antioxidant. The water-soluble antioxidant may be added to the medium to prevent oxidation damage. Examples of suitable water-soluble antioxidants include curcumin, glutathione, uric acid, Trolox or Allicidin to mention only a few.

The present invention relates, in a third aspect, to a method of treating rosacea, psoriasis, eczema, dermatitis, topical steroid withdrawal syndrome, epidermolysis bullosa, or skin damage caused by fragile skin, wherein the method comprises topically administering conditioned medium derived from cultivation of mesenchymal stem cells of the umbilical cord to the skin of a subject.

For treating the subject, the conditioned medium of the invention is administered herein by topical administration. In principle any way of topical administration is meant herein. For example, it is possible to formulate the conditioned medium into a cream, ointment, gel, lotion, suspension or any other suitable formulation before applying the conditioned medium to the skin of the subject. The conditioned medium may also or alternatively be held in place, e.g., by a dressing or by soaking the conditioned medium into a gauze which is then applied to the skin of the subject (see Example 3).

Conditioned medium or a composition comprising the conditioned medium as described herein and in accordance of the present invention is administered topically to the skin of a subject surrounding the wound or the damaged skin area. Thus, the method does not comprise administering the conditioned medium onto the wound or the damaged skin area. The skin surrounding a wound or damaged skin area, in some examples, is the skin immediately surrounding the wound or damaged skin. In some examples, the skin surrounding the wound refers to the periwound area which is the skin beyond the wound edge but close or adjacent to the wound edge. In some embodiments, the skin to be treated is the intact skin. Particularly, the skin surrounding the wound may refer to intact periwound skin that can perform the regular functions of skin, such as absorption, excretion, protection, secretion, thermoregulation and immunity to name just a few, and that maintains the physical characteristics of skin such as elasticity and surface integrity. In some embodiments, the skin to be treated according to the present invention is thin skin/weak skin. As used herein, weak skin preferably refers to particularly vulnerable and/or fragile skin that has been impacted by a wound or damaged skin but is still intact, i.e., can perform the regular functions of skin and maintains its physical characteristics as outlined above. The term “thin skin” is used herein in its regular meaning to refer to skin that tears, bruises, or breaks easily. Thin skin is sometimes called thinning skin, or fragile skin. Thin skin is a common condition in older adults and is most noticeable in the face, arms, and hands. A person with thin skin may find that they are able to see the veins, tendons, bones, and capillaries under the skin of their hands and arms. Thin skin is however not only associated with aging but also the cause of disease such as diabetes, the use of certain medications such as steroids or can also be caused by UV exposure, genetics, or lifestyle, for example. The thick, fibrous tissue of the dermis is made of collagen and elastin. The dermis provides strength, flexibility, and elasticity to the skin. Thin skin is the result of the thinning of the dermis.

In some embodiments, the conditioned medium is administered in the form of a gel. Particularly, the present invention describes gel formulations comprising the conditioned medium for a topical application wherein the formulation comprises an effective amount of conditioned medium for inducing, stimulating, and/or promoting wound healing or healing of a damaged skin area and/or for preventing formation and/or recurrence of such a wound or damaged skin area and/or for treating a disease associated with a wound or skin damage as described herein. A gel formulation as used herein refers to a gel that contains the conditioned medium of the present invention in an amount effective for healing a wound or healing a damaged skin area and/or for preventing formation and/or recurrence of such a wound or damaged skin area and/or for treating a disease associated with skin damage or a wound as described herein and, additionally, can contain water soluble, pharmaceutically and/or cosmetically accepted polymers such as polyacrylic acids (carbomers). Such gelling agents may provide a suitable viscosity of the gel such as within the range for topical applications such as 1000-200,000 cps at room temperature. Gel formulations have the advantage of uniform spreading on the surrounding area of a wound or damaged skin area which increases the contact time of the skin with active components of the formulation, such active components comprising for example growth factors and/or other biological factors, and delivers the active components of the formulation over time. The conditioned medium of the present invention in the form of a gel may particularly be used in a hospital setting, i.e., for treating a wound or a damaged skin area of a patient who requires treatment of the wound or damaged skin area. Thus, the conditioned medium in the form of a gel may be particularly useful for treating patients at the hospital suffering from a wound or a damaged skin area wherein the wound or damaged skin area developed prior to or during hospital stay. In illustrative examples such a wound may be diabetic foot ulcer or a pressure ulcer wherein the pressure ulcer developed due to prolonged pressure exerted over specific areas of the body.

In some embodiments, the conditioned medium is administered in the form of a liquid such as a lotion or ointment. The liquid formulation as used herein refers to a solution or liquid preparation that contains the conditioned medium of the present invention in an amount effective for healing a wound or healing a damaged skin area and/or for preventing formation and/or recurrence of such a wound or damaged skin area and/or for treating a skin disease associated with a wound or damaged skin. The conditioned medium of the present invention in the form of a liquid may be particularly useful for the administration in a home setting, i.e., the liquid formulation comprising the conditioned medium of the present invention may be administered without the need of a doctor. For example, the conditioned medium in the form of a liquid may be administered by a subject who is at risk of developing a wound or a damaged skin area but who is not hospitalized or does not need to stay at a hospital. For example, the conditioned medium in the form of a liquid may be applied as a “daily skin conditioner” directly by a subject being at risk of developing a diabetic ulcer, wherein the subject is at home and not being treated in a hospital or wound care center.

The present invention, in a fourth aspect, relates to a method of producing a conditioned medium. This medium can then subsequently be used for a cosmetic or therapeutic application as described herein. This method may comprise:
(a) cultivating mesenchymal stem cells of the umbilical cord in a culture medium comprising DMEM (Dulbecco's modified eagle medium), F12 (Ham's F12 Medium), M171 (Medium 171) and FBS (Fetal Bovine Serum);
(b) removing the mesenchymal stem cells of the umbilical cord from the culture medium;
(c) optionally cultivating the mesenchymal stem cells of the umbilical cord in a further culture medium, optionally wherein the further cell culture medium comprises a water-soluble antioxidant, wherein the conditioned medium is obtained by collecting the cell culture medium.

The conditioned medium may be derived, after cultivation of the mesenchymal stem cells in the medium that comprises DMEM, F12, M171 and FBS, from cultivation of the mesenchymal stem cells of the umbilical cord in the above-mentioned further culture medium. This further culture medium may be any (further) basic culture medium suitable for animal cell growth, and in particular a basal medium suitable for cultivation of mesenchymal stem cells. In some examples, the further cell culture medium does not comprise a growth factor and/or is a serum-free medium and/or wherein the cells are cultivated at a concentration of about 1 million cells per 1 ml. In some examples, the further culture medium may be a serum-free medium. Non-limiting examples of such a serum-free medium include Minimal Essential Medium (MEM), Dulbecco modified Eagle Medium (DMEM), Roswell Park Memorial Institute Medium (RPMI), and Keratinocyte Medium (KM), KBM (Keratinocyte Basal medium), EpiLife KM (Keratinocyte-EpiLife medium) etc. The further culture medium may alternatively also contain serum. As an example of such a serum containing medium, the conditioned medium to be used may be one that is described and usually used for the isolation and cultivation of a mesenchymal stem cell population of the amniotic membrane of umbilical cord such as medium PTT-4. This medium PTT-4 consists of 90% (v/v) CMRL1066, and 10% (v/v) FBS) and has been used in US patent application US 2008/0248005 and the corresponding International patent application WO2007/046775 for the isolation and cultivation of a mesenchymal stem cell population of the amniotic membrane of umbilical cord which was shown to have excellent wound healing properties in US patent application US 2008/0248005 and International patent application WO2007/046775. In some examples, a cell culture medium as described herein such as a culture medium comprising DMEM (Dulbecco's modified eagle medium), F12 (Ham's F12 Medium), M171 (Medium 171) and FBS (Fetal Bovine Serum) may be used for cultivation of mesenchymal stem cells of the umbilical cord, wherein the cell culture medium is removed after cultivation, e.g., once the cells reach full confluency, and replaced by another culture medium/solution such as a culture medium solution comprising, for example, DMEM which may comprise an antioxidant ingredient. Generally, during the optional cultivation step in the further medium of the method of preparing a conditioned medium as described here, the mesenchymal stem cells can be cultivated for any suitable period of time. Such a suitable period of time may, for example, be a period of 1 day, 2 days, 3 days, 4 days, 5 days, 6 days, 7 days or even longer, for example, a period of up to 10 days or 14 days, if desired. In this context, it is noted for clarity, that both, the conditioned medium obtained in the first cultivation step and also the conditioned medium obtained in the second (further) cultivation step, can be used for inducing, stimulating and/or promoting healing of a wound or damaged skin area and/or for preventing the formation and/or recurrence of a wound or damaged skin and/or for treating a disease associated with a wound or damaged skin as described herein.

The mesenchymal stem cells of the umbilical cord (tissue) used herein for the preparation of conditioned medium may be (derived) from any compartment of umbilical cord tissue that contains mesenchymal stem cells. The mesenchymal stem cell population may be a mesenchymal stem cell population of the amnion (AM), a perivascular (PV) mesenchymal stem cell population, a mesenchymal stem cell population of Wharton's jelly (WJ), a mesenchymal stem cell population of the amniotic membrane of umbilical cord (also referred to as cord lining mesenchymal cells) but also a mixed mesenchymal stem cell population of the umbilical cord (MC), meaning a population of mesenchymal stem cells that includes stem cells of two or more of these compartments. In some examples, the mesenchymal stem cells are of the amniotic membrane of the umbilical cord. In some examples, the mesenchymal stem cells of the umbilical cord comprise mesenchymal stem cells of the amniotic membrane of the umbilical cord. The mesenchymal stem cells of these compartments and the isolation therefrom are known to the person skilled in the art and are described, for example, by Subramanian et al “Comparative Characterization of Cells from the Various Compartments of the Human Umbilical Cord Shows that the Wharton's Jelly Compartment Provides the Best Source of Clinically Utilizable Mesenchymal Stem Cells”, PLOS ONE 10(6): e0127992, 2015 and the references cited therein, Van Pham et al. “Isolation and proliferation of umbilical cord tissue derived mesenchymal stem cells for clinical applications”, Cell Tissue Bank (2016) 17:289-302, 2016. A mixed mesenchymal stem cell population of the umbilical cord can, for example, be obtained by removing the arteries and veins from the umbilical cord tissue, cutting the remaining tissue and the Wharton's jelly into piece and cultivating the umbilical cord tissue (by tissue explant) in a culture medium, such as PTT-6. A mixed mesenchymal stem cell population of the umbilical cord may also be obtained by culturing entire umbilical cord tissue with intact umbilical vessels as tissue explant under the conditions (cultivation in serum-supplemented DMEM with 10% fetal bovine serum, 10% horse serum, and 1% Penicillin/Streptomycin) as described by Schugar et al. “High harvest yield, high expansion, and phenotype stability of CD146 mesenchymal stromal cells from whole primitive human umbilical cord tissue. Journal of biomedicine & biotechnology. 2009; 2009:789526”. In this context, it is noted that a mesenchymal stem cell population of the cord-placenta junction can be isolated as described by Beeravolu et al. “Isolation and Characterization of Mesenchymal Stromal Cells from Human Umbilical Cord and Fetal Placenta.” J Vis Exp. 2017; (122): 55224.

In accordance with the above, it is noted here that conditioned medium is derived from cultivation of mesenchymal stem cells of umbilical cord tissue, wherein the stem cells can be cultivated in a culture medium comprising DMEM (Dulbecco's modified eagle medium), F12 (Ham's F12 Medium), M171 (Medium 171) and FBS (Fetal Bovine Serum) and which can be isolated from its natural environment prior to cultivation in a culture medium as described herein. Such an approach is in particular used for a mesenchymal stem cell population of the umbilical cord. Such a stem cell population, say a mesenchymal stem cell population of Wharton's jelly, may first be isolated as described above by Subramanian et al, 2015, PLOS ONE, supra or International Patent application WO 2004/072273 “Progenitor Cells From Wharton's Jelly Of Human Umbilical Cord” and then be subjected to cultivation of the isolated mesenchymal stem cell population in the culture medium that comprises DMEM (Dulbecco's modified eagle medium), F12 (Ham's F12 Medium), M171 (Medium 171) and FBS (Fetal Bovine Serum). Also a placental mesenchymal stem cell population may be isolated from placenta as described in European patent application EP1 288 293, Talwadekar et al, “Cultivation and Cryopreservation of Cord Tissue MSCs with Cord Blood AB Plasma” Biomed Res J 2014; 1(2): 126-136, Talwadekar et al, “Placenta-derived mesenchymal stem cells possess better immunoregulatory properties compared to their cord-derived counterparts—a paired sample study” Scientific Reports 5:15784 (2015), or Beeravolu et al. “Isolation and Characterization of Mesenchymal Stromal Cells from Human Umbilical Cord and Fetal Placenta.” J Vis Exp. 2017; (122): 55224, for example, and subsequently cultivated in the culture medium as described herein.

In this context, it is noted that the culture medium as described herein therefore also allows the isolation of a mesenchymal stem cell population (also referred here as “mesenchymal stem cells”) from its natural environment. Accordingly, the culture medium as described herein allows also isolation of a mesenchymal stem cell population under conditions that allow cell proliferation of the mesenchymal stem/progenitor cells without differentiation of the mesenchymal stem/progenitor cells. In some examples, the cell culture medium(s) used for isolation and/or cultivation of mesenchymal stem cells of the umbilical cord may be the medium used for cultivation of the cells in order to produce the conditioned medium of the present invention.

In accordance with the above disclosure, it is noted here that such a mesenchymal stem cell population described herein can be isolated and cultivated (i.e., are derived) from any umbilical cord tissue as long as the umbilical cord tissue contains the amniotic membrane (which is also referred to as “cord lining”). Accordingly, the mesenchymal stem cell population can be isolated from (pieces of) the entire umbilical cord as described in the experimental section of the present application. This umbilical cord tissue may thus contain, in addition to the amniotic membrane, any other tissue/component of the umbilical cord. As shown, for example, in FIG. 16 of US patent application 2006/0078993 or International patent application WO 2006/019357, the amniotic membrane of the umbilical cord is the outmost part of the umbilical cord, covering the cord. In addition, the umbilical cord contains one vein (which carries oxygenated, nutrient-rich blood to the fetus) and two arteries (which carry deoxygenated, nutrient-depleted blood away from the fetus). For protection and mechanical support these three blood vessels are embedded in the Wharton's jelly, a gelatinous substance made largely from mucopolysaccharides. Accordingly, the umbilical cord tissue used herein can also comprise this one vein, the two arteries and the Wharton's jelly. The use of such an entire (intact) section of the umbilical cord has the advantage that the amniotic membrane does not need to be separated from the other components of the umbilical cord. This reduces the isolation steps and thus makes the method of the present invention, simpler, faster, less error prone and more economical. The isolation of the mesenchymal stem cells can thus start by tissue explant, which may be followed by subsequent subculturing (cultivation) of the isolated mesenchymal stem cells if greater amounts of the mesenchymal stem cells are desired. Alternatively, it is also possible to first separate the amniotic membrane from the other components of the umbilical cord and isolate the mesenchymal cord lining stem cells from the amniotic membrane by cultivation of the amniotic membrane in a culture medium as described herein. This cultivation can also be carried out by tissue explant, optionally followed by subculturing of the isolated mesenchymal stem cells.

In this context, the term “tissue explant” or “tissue explant method” is used in its regular meaning in the art to refer a method in which a tissue (for example, umbilical cord tissue), once being harvested, or a piece of the tissue is being placed in a cell culture dish containing culture (growth) medium and by which over time, the stem cells migrate out of the tissue onto the surface of the dish. These primary stem cells can then be further expanded and transferred into fresh dishes through micropropagation (subculturing) as also described here. In this context, it is noted that in the first step of isolating/obtaining a mesenchymal stem cell population of the umbilical cord such as amniotic membrane or Wharton's jelly mesenchymal stem cells, a master cell bank of the isolated mesenchymal stem cells is obtained, while in the subsequent subculturing a working cell bank can be obtained. If conditioned medium derived from cultivation of mesenchymal stem cells of the umbilical cord of the invention is used for application, e.g., in promoting healing of a wound, a cell population of the working cell bank will be typically used for this purpose. Both the stem cell population of the isolation step (which may make up the master cell bank) and the stem cell population of the subculturing step (which may make up the working cell bank) can, for example, be stored in cryopreserved form.

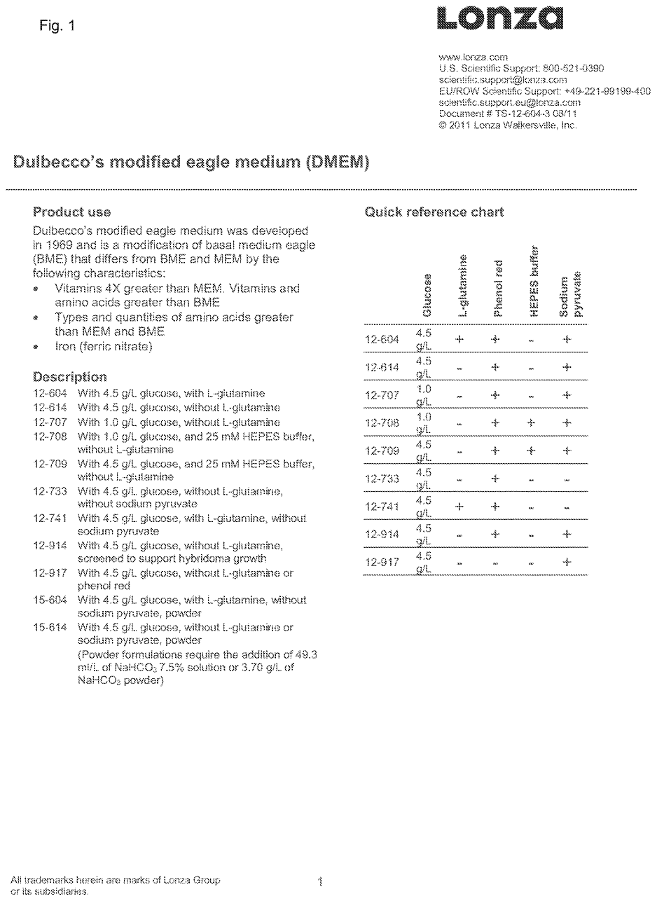

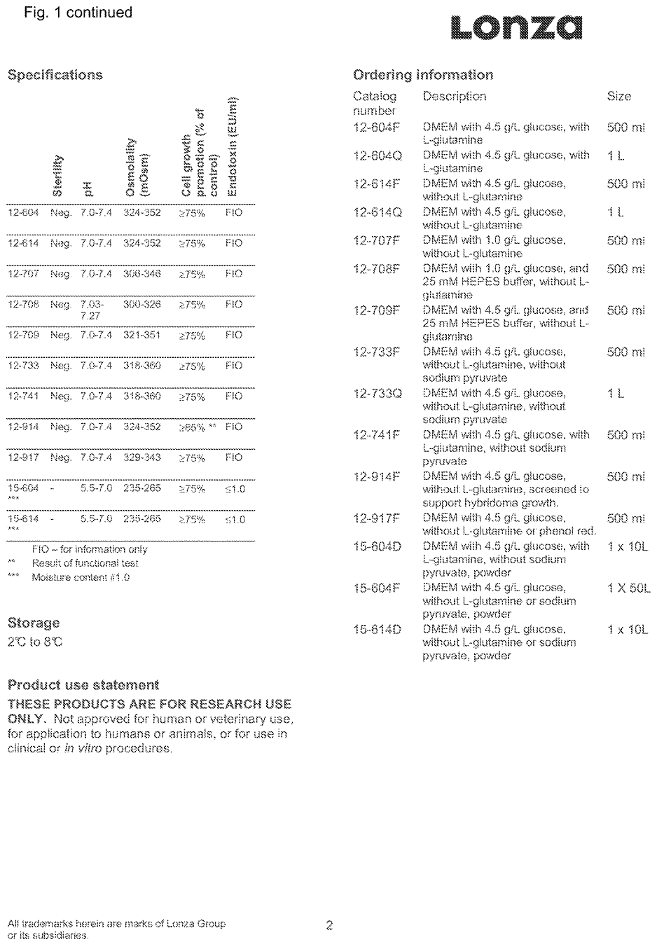

By “DMEM” is meant Dulbecco's modified eagle medium which was developed in 1969 and is a modification of basal medium eagle (BME) (cf. FIG. 1 showing the data sheet of DMEM available from Lonza). The original DMEM formula contains 1000 mg/L of glucose and was first reported for culturing embryonic mouse cells. DMEM has since then become a standard medium for cell culture that is commercially available from various sources such as ThermoFisher Scientific (catalogue number 11965-084), Sigma Aldrich (catalogue number D5546) or Lonza, to name only a few suppliers. Thus, any commercially available DMEM can be used in the present invention. In preferred embodiments, the DMEM used herein is the DMEM medium available from Lonza under catalog number 12-604F. This medium is DMEM supplemented with 4.5 g/L glucose and L-glutamine). In another preferred embodiment the DMEM used herein is the DMEM medium of Sigma Aldrich catalogue number D5546 that contains 1000 mg/L glucose, and sodium bicarbonate but is without L-glutamine.

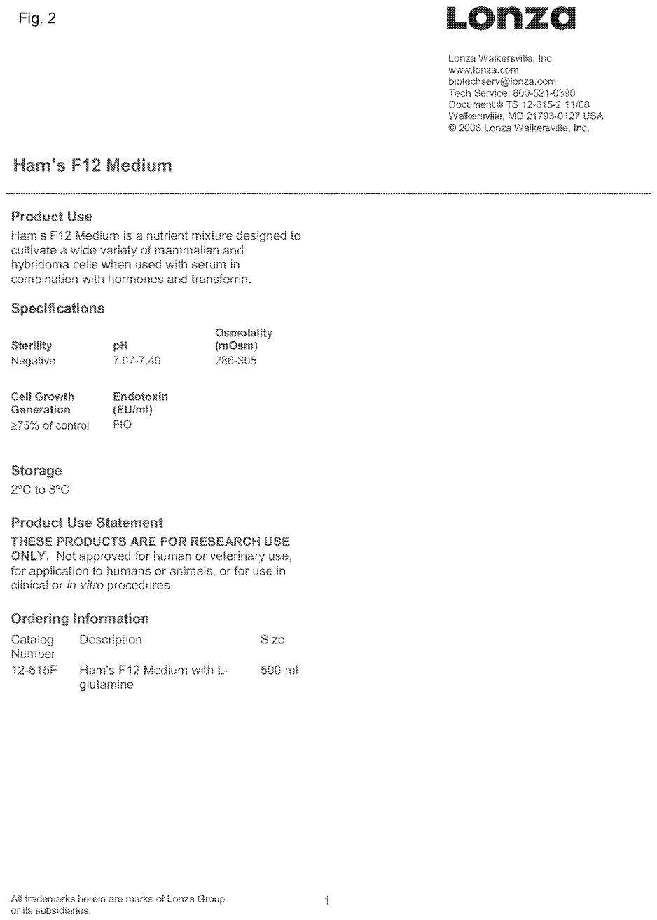

By “F12” medium is meant Ham's F12 medium. This medium is also a standard cell culture medium and is a nutrient mixture initially designed to cultivate a wide variety of mammalian and hybridoma cells when used with serum in combination with hormones and transferrin (cf. FIG. 2, showing the data sheet of Ham's F12 medium from Lonza). Any commercially available Ham's F12 medium (for example, from ThermoFisher Scientific (catalogue number 11765-054), Sigma Aldrich (catalogue number N4888) or Lonza, to new only a few suppliers) can be used in the present invention. In preferred embodiments, Ham's F12 medium from Lonza is used.

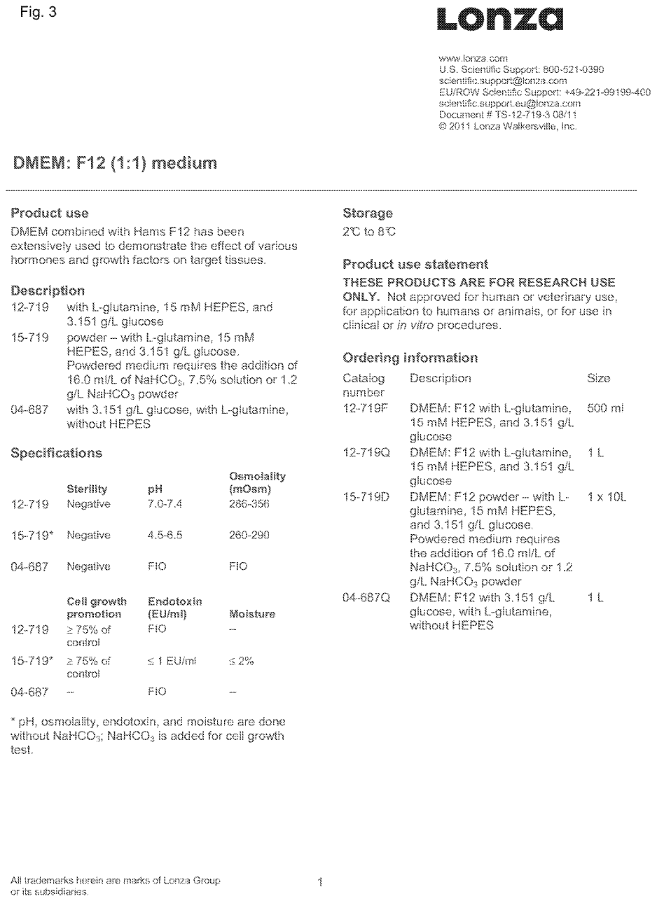

By “DMEM/F12” or “DMEM:F12” is meant a 1:1 mixture of DMEM with Ham's F12 culture medium (cf. FIG. 3 showing the data sheet for DMEM:F12 (1:1) medium from Lonza). Also DMEM/F12 (1:1) medium is a widely used basal medium for supporting the growth of many different mammalian cells and is commercially available from various supplier such as ThermoFisher Scientific (catalogue number 11330057), Sigma Aldrich (catalogue number D6421) or Lonza. Any commercially available DMEM:F12 medium can be used in the present invention. In preferred embodiments, the DMEM:F12 medium used herein is the DMEM/F12 (1:1) medium available from Lonza under catalog number 12-719F (which is DMEM:F12 with L-glutamine, 15 mM HEPES, and 3.151 g/L glucose).

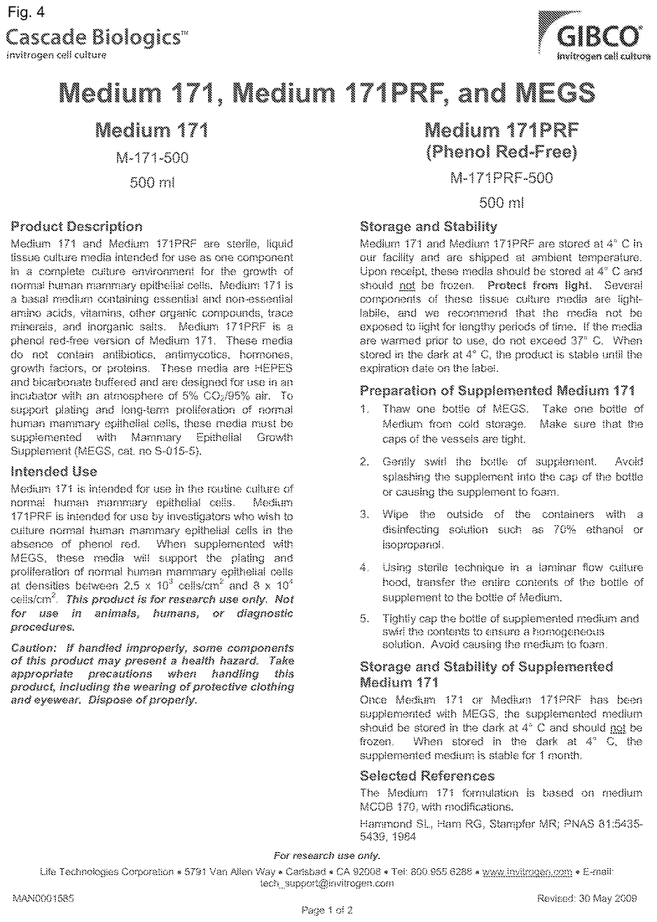

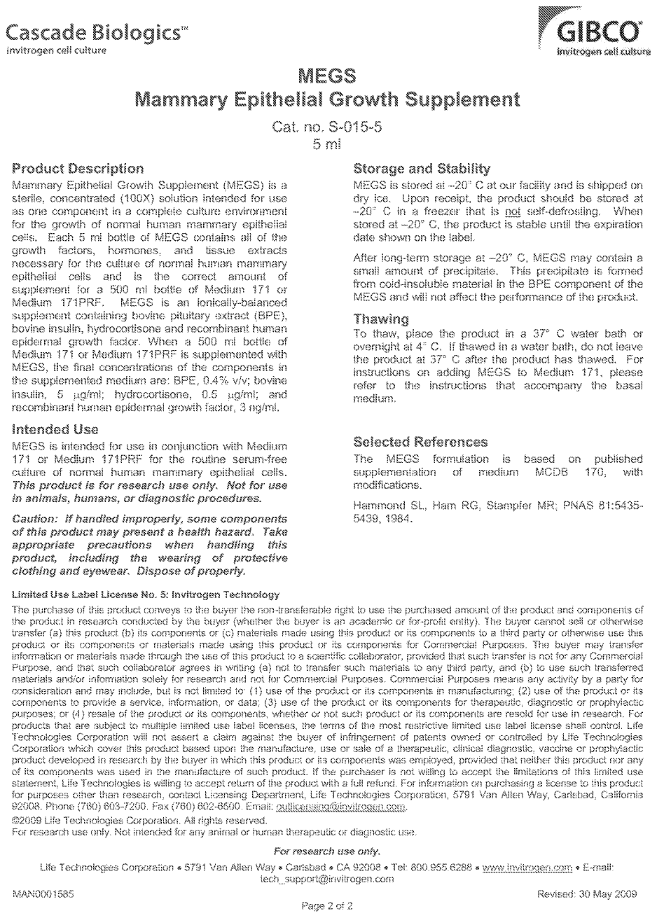

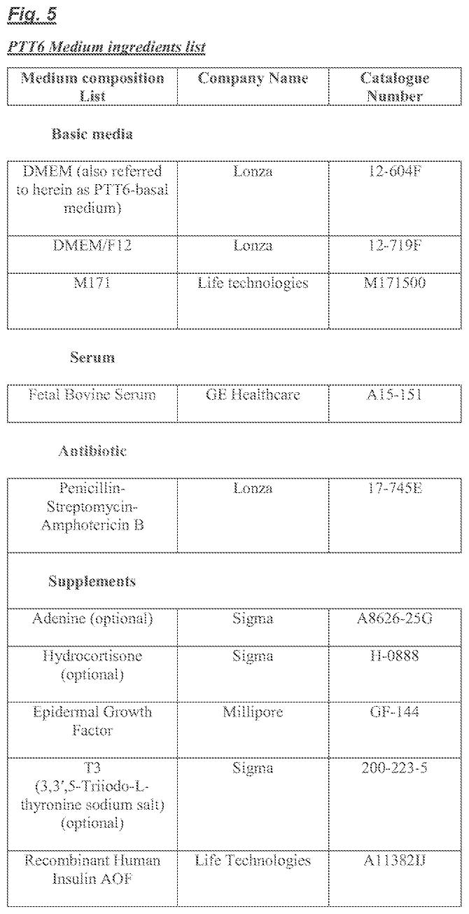

By “M171” is meant culture medium 171, which has been developed as basal medium for the culture of for the growth of normal human mammary epithelial cells (cf. FIG. 4 showing the data sheet for M171 medium from Life Technologies Corporation). Also this basal medium is widely used and is commercially available from supplier such as ThermoFisher Scientific or Life Technologies Corporation (catalogue number M171500), for example. Any commercially available M171 medium can be used in the present invention. In preferred embodiments, the M171 medium used herein is the M171 medium available from Life Technologies Corporation under catalogue number M171500.

By “FBS” is meant fetal bovine serum (that is also referred to as “fetal calf serum”), i.e., the blood fraction that remains after the natural coagulation of blood, followed by centrifugation to remove any remaining red blood cells. Fetal bovine serum is the most widely used serum-supplement for in vitro cell culture of eukaryotic cells because it has a very low level of antibodies and contains more growth factors, allowing for versatility in many different cell culture applications. The FBS is preferably obtained from a member of the International Serum Industry Association (ISIA) whose primary focus is the safety and safe use of serum and animal derived products through proper origin traceability, truth in labeling, and appropriate standardization and oversight. Suppliers of FBS that are ISIA members include Abattoir Basics Company, Animal Technologies Inc., Biomin Biotechnologia LTDA, GE Healthcare, Gibco by Thermo Fisher Scientific and Life Science Production, to mention only a few. In currently preferred embodiments, the FBS is obtained from GE Healthcare under catalogue number A15-151.

The culture medium as described herein may comprise for deriving the conditioned medium from cultivation of the mesenchymal stem cells of the umbilical cord DMEM in a final concentration of about 55 to 65% (v/v), F12 in a final concentration of about 5 to 15% (v/v), M171 in a final concentration of about 15 to 30% (v/v) and FBS in a final concentration of about 1 to 8% (v/v). The value of “% (v/v)” as used herein refers to the volume of the individual component relative to the final volume of the culture medium. This means, if DMEM is, for example, present in the culture medium a final concentration of about 55 to 65% (v/v), 1 liter of culture medium contains about 550 to 650 ml DMEM. In other embodiments, the culture medium may comprise DMEM in a final concentration of about 57.5 to 62.5% (v/v), F12 in a final concentration of about 7.5 to 12.5% (v/v), M171 in a final concentration of about 17.5 to 25.0% (v/v) and FBS in a final concentration of about 1.75 to 3.5% (v/v). In further embodiments, the culture medium may comprise DMEM in a final concentration of about 61.8% (v/v), F12 in a final concentration of about 11.8% (v/v), M171 in a final concentration of about 23.6% (v/v) and FBS in a final concentration of about 2.5% (v/v).

In addition to the above-mentioned components, the culture medium may comprise supplements that are advantageous for cultivation of the mesenchymal stem cells of the umbilical cord and for deriving conditioned medium for inducing, stimulating and/or promoting healing of a wound or damaged skin and/or for preventing formation and/or recurrence of a wound or skin damage and/or for treating a wound or damaged skin. The culture medium as described herein may, for example, comprise Epidermal Growth Factor (EGF). If present, EGF may be present in the culture medium in a final concentration of about 1 ng/ml to about 20 ng/ml. In some of these embodiments, the culture medium may comprise EGF in a final concentration of about 10 ng/ml.

The culture medium as described herein may also comprise insulin. If present, insulin may be present in a final concentration of about 1 μg/ml to 10 μg/ml. In some of these embodiments, the culture medium may comprise Insulin in a final concentration of about 5 μg/ml.

The culture medium may further comprise at least one of the following supplements: adenine, hydrocortisone, and 3,3′,5-Triiodo-L-thyronine sodium salt (T3). In such embodiments, the culture medium may comprise all three of adenine, hydrocortisone, and 3,3′,5-Triiodo-L-thyronine sodium salt (T3). In these embodiments, the culture medium may comprise adenine in a final concentration of about 0.05 to about 0.1 μg/ml adenine, hydrocortisone in a final concentration of about 1 to about 10 μg/ml hydrocortisone and/or 3,3′,5-Triiodo-L-thyronine sodium salt (T3) in a final concentration of about 0.5 to about 5 ng/ml.

As described herein, umbilical cord tissue may be cultured till a suitable number of (primary) mesenchymal stem cells such as cord lining stem cells, Wharton's Jelly or placental stem cells have outgrown from the tissue. Typically, the umbilical cord tissue is cultivated until cell outgrowth of the mesenchymal stem cells of the respective tissue reaches about 70 to about 80% confluency. It is noted here that the term “confluency” or “confluence” is used in its regular meaning in the art of cell culture and is meant as an estimate/indicator of the number of adherent cells in a culture dish or a flask, referring to the proportion of the surface which is covered by cells. For example, 50 percent confluence means roughly half of the surface is covered and there is still room for cells to grow. 100 percent confluence means the surface is completely covered by the cells, and no more room is left for the cells to grow as a monolayer.

Once a suitable number of primary cells (mesenchymal stem cells) have been obtained from the respective tissue by tissue explant, the mesenchymal stem cells are removed from the cultivation container used for the cultivation. By so doing, a master cell bank containing the (primary) isolated mesenchymal stem cells of for example, the umbilical cord or the placenta can be obtained. Typically, since such mesenchymal stem cells are adherent cells, harvesting the cells is carried out using standard enzymatic treatment. For example, the enzymatic treatment may comprise trypsination as described in International US patent application 2006/0078993, International patent application WO2006/019357 or International patent application WO2007/046775, meaning outgrowing cells can be harvested by trypsinization (0.125% trypsin/0.05% EDTA) for further expansion. If the harvested mesenchymal stem cells are, for example, used for generating a master cell bank, the cells can also be cryo-preserved and stored for further use as explained herein below.

Once being harvested, the mesenchymal stem cells can be transferred to a cultivation container for subculturing. Subculturing or culturing (both terms are used interchangeable hereinafter) will be also be carried out if a mesenchymal stem cell population of the umbilical cord is employed that has been isolated from its natural environment earlier. The subculturing can also be started from frozen primary cells, i.e., from the master cell bank. For subculturing any suitable amount of cells can be seeded in a cultivation container such as cell culture plate. The mesenchymal cells can, for this purpose, be suspended in a suitable medium (most conveniently, the culture medium as described herein) for subculturing at a concentration of, for example, about 0.5×10⁶ cells/ml to about 5.0×10⁶ cells/ml. For example, the cells are suspended for subcultivation at a concentration of about 1.0×10⁶ cells/ml. The subculturing can be carried by cultivation either in simple culture flasks but also, for example, in a multilayer system such as CellStacks (Corning, Corning, NY, USA) or Cellfactory (Nunc, part of Thermo Fisher Scientific Inc., Waltham, MA, USA) that can be stacked in incubators. Alternatively, the subculturing can also be carried out in a closed self-contained system such as a bioreactor. Different designs of bioreactors are known to the person skilled in the art, for example, parallel-plate, hollow-fiber, or micro-fluidic bioreactors. See, for example, Sensebe et al. “Production of mesenchymal stromal/stem cells according to good manufacturing practices: a review”, supra. An illustrative example of a commercially available hollow-fiber bioreactor is the Quantum® Cell Expansion System (Terumo BCT, Inc). that has, for example, been used for the expansion of bone marrow mesenchymal stem cells for clinical trials (cf., Hanley et al, Efficient Manufacturing of Therapeutic Mesenchymal Stromal Cells Using the Quantum Cell Expansion System, Cytotherapy. 2014 August; 16 (8): 1048-1058). Another example of commercially available bioreactors that can be used for the subculturing of the mesenchymal stem cell population as described herein is the Xuri Cell Expansion System available from GE Heathcare. The cultivation of the mesenchymal stem cell population in an automated system such as the Quantum® Cell Expansion System is of particular benefit if a working cell bank is to be produced under GMP conditions and a high number of cells is wanted.

The subculturing of the mesenchymal stem cells of the invention can take place in a culture medium as described herein. Accordingly, the culture medium can be used both for the isolation of the mesenchymal stem cell population, for example, from the amniotic membrane or from Wharton's jelly of umbilical cord and the subsequent cultivation of the isolated primary cells by cultivation. Also for the cultivation, the mesenchymal stem cells can be cultured till a suitable amount of cells have grown. In some examples, the mesenchymal stem cells are cultured till the mesenchymal stem cells reach about 70% confluency, about 80% confluency, about 90% confluency, about 95% confluency, to about 96% or even 100% confluency.

The isolation/cultivation of the population of mesenchymal stem cell population can be carried out under standard condition for the cultivation of mammalian cells. Typically, the method of isolating the population of the mesenchymal stem cells is carried out under conditions (temperature, atmosphere) that are normally used for cultivation of cells of the species of which the cells are derived. For example, human umbilical cord tissue and the mesenchymal cord lining stem cells, respectively, are usually cultivated at 37° C. in normal atmosphere with 5% CO₂. In this context, it is noted that the mesenchymal cell population as described herein may be derived of any mammalian species, such as human, red deer, mouse, rat, guinea pig, pig, rabbit, goat, horse, dog, cat, sheep, or monkey. In illustrative embodiments, mesenchymal stem cells of the umbilical cord are of red deer or human.

Once a desired/suitable number of mesenchymal stem cells have been obtained from the culture or subculture, the mesenchymal stem cells are harvested by removing them from the cultivation container used for the cultivation. The harvesting of the mesenchymal stem cells is typically again carried out by enzymatic treatment, including comprises trypsination of the cells. The isolated mesenchymal stem cells are subsequently collected and are either be directedly used or preserved for further use. Typically, preserving is carried out by cryo-preservation. The term “cryo-preservation” is used herein in its regular meaning to describe a process where the mesenchymal stem cells are preserved by cooling to low sub-zero temperatures, such as (typically) −80° C. or −196° C. (the boiling point of liquid nitrogen). Cryo-preservation can be carried out as known to the person skilled in the art and can include the use of cryo-protectors such as dimethylsulfoxide (DMSO) or glycerol, which slow down the formation of ice-crystals in the cells of the umbilical cord.

The isolated population of the mesenchymal stem cells that is obtained by the cultivation and/or isolation method as described herein is highly defined and homogenous. Such a highly homogenous population of mesenchymal stem cells derived from tissue such as the amniotic membrane or Wharton's Jelly of the umbilical cord has been reported in WO 2018/067071 A1 and WO 2019/199234 A1. For example, as disclosed and shown in the experimental section in WO 2019/199234 A1A1, at least about 90%, at least about 91% or more, about 92% or more, about 93% or more, about 94% or more, about 95% or more, about 96% or more, about 97% or more, about 98% or more about 99% or more cells of the mesenchymal stem cell population of the umbilical cord (for example, isolated from Wharton's jelly or the amniotic membrane of the umbilical cord) express each of the following markers: CD73, CD90 and CD105 and lack expression of CD34, CD45 and HLA-DR, meaning that this percentage of the isolate cell population express each of CD73, CD90 and CD105 and lack expression of the following markers: CD34, CD45 and HLA-DR. Such a highly homogenous population of mesenchymal stem cells derived from the amniotic membrane of the umbilical cord meets the criteria for mesenchymal stem cells to be used for cellular therapy (also cf. the Experimental Section in WO 2019/199234 A1 and, for example, Sensebe et al. “Production of mesenchymal stromal/stem cells according to good manufacturing practices: a review”, supra). It is noted in this context that this mesenchymal stem cell population can be obtained by either the isolating method as described in WO 2019/199234 A1 but also by a different method such as cell sorting, if wanted.

In a further aspect the invention is directed to a method of inducing, stimulating and/or promoting wound healing and/or preventing formation and/or recurrence of a wound and/or treating rosacea, psoriasis, eczema, dermatitis, topical steroid withdrawal syndrome, epidermolysis bullosa, or skin damage caused by fragile skin, wherein the method comprises treating the skin of a subject surrounding the wound or the damaged skin area or the intact skin of a subject being at risk of developing a wound or a damaged skin area with conditioned medium derived from cultivation of mesenchymal stem cells of the umbilical cord in a culture medium as described herein, wherein the culture medium is mixed as follows to obtain a final volume of 500 ml culture medium:
i. 250 ml of DMEM
ii. 118 ml M171
iii. 118 ml DMEM/F12
iv. 12.5 ml Fetal Bovine Serum (FBS) to reach a final concentration of 2.5% (v/v).

As explained above, DMEM/F12 medium is a 1:1 mixture of DMEM and Ham's F12 medium. Thus, 118 ml DMEM/F12 medium contain 59 ml DMEM and 59 ml F12. Accordingly, when using this method of making a culture medium, the final concentrations (v/v) with 500 ml total volume are as follows:
DMEM: 250 ml+59 ml=309 ml, corresponds to 309/500=61.8% (v/v)
M171: 118 ml, corresponds to 118/500=23.6% (v/v)
F12: 59 ml, corresponds to 59/500=11.8% (v/v).

The culture medium as described herein may further comprise adding v. 1 ml EGF stock solution (5 μg/ml) to achieve a final EGF concentration of 10 ng/ml, and vi. Insulin 0.175 ml stock solution (14.28 mg/ml) to achieve a final insulin concentration of 5 μg/ml.

It is noted here that the above-mentioned volumes of these components i. to vi when mixed result in a final volume of 499.675 ml culture medium. If no further components are added to the culture medium, the remaining 0.325 ml (to add up to a volume of 500 ml) can, for example, be any of components i. to iv, that means either DMEM, M171, DMEM/F12 or FBS. Alternatively, the concentration of the stock solution of EGF or Insulin can of course be adjusted such that the total volume of the culture medium is 500 ml. In addition, it is also noted that components i. to iv. do not necessarily have to be added in the order in which they are listed but it is of course also possible to use any order to mix these components to arrive at the culture medium as described herein. This means, that for example, M171 and DMEM/F12 can be mixed together and then combined with DMEM and FBS to reach final concentrations as described here, i.e., a final concentration of DMEM of about 55 to 65% (v/v), a final concentration of F12 of about 5 to 15% (v/v), a final concentration of M171 of about 15 to 30% (v/v) and a final concentration of FBS of about 1 to 8% (v/v).

In other embodiments, the method further comprises adding to DMEM a volume of 0.325 ml of one or more of the following supplements: adenine, hydrocortisone, 3,3′,5-Triiodo-L-thyronine sodium salt (T3), thereby reaching a total volume of 500 ml culture medium. In this embodiment, the final concentration of these supplements in DMEM may be as follows:
about 0.05 to 0.1 μg/ml adenine, for example about 0.025 μg/ml adenine,
about 1 to 10 g/ml hydrocortisone,
about 0.5 to 5 ng/ml 3,3′,5-Triiodo-L-thyronine sodium salt (T3), for example 1.36 ng/ml 3,3′,5-Triiodo-L-thyronine sodium salt (T3).

In some embodiments, conditioned medium of the present invention is produced by culturing mesenchymal stem cells of the umbilical cord in a cell culture medium as described herein, such as in a culture medium comprising DMEM (Dulbecco's modified eagle medium), F12 (Ham's F12 Medium), M171 (Medium 171) and FBS (Fetal Bovine Serum), removing the mesenchymal stem cells of the umbilical cord from the culture medium, wherein the conditioned medium is obtained by collecting the cell culture medium. Additionally, in some examples, the culture medium in which the stem cells have been cultivated may be replaced by and the cells may be cultivated in a second cell culture medium, which may be the same or a different cell culture medium than the first one. In some examples, the second cell culture medium may be a growth factor-free medium and/or a serum-free medium. Alternatively, mesenchymal stem cells of the umbilical cord that have been previously cultured (e.g., cultured stem cells that have been stored) can be further cultivated in growth factor-free and/or serum-free medium. In either case, the resultant medium is referred to as conditioned medium. The resultant conditioned medium is harvested, and may be further processed to produce a composition comprising conditioned medium of the present invention.

Conditioned medium of the present invention or a composition comprising the conditioned medium of the present invention can be diluted in any suitable diluent/carrier medium as long as the carrier medium does not affect function and safety of the conditioned medium when topically administered on the skin of a subject. For example, conditioned medium may be diluted in PBS, water, a basal medium or a cream formulation. A basal medium as used herein refers to a mixture containing saccharides, amino acids, water, etc., required for the survival of a cell and include commercial prepared medium such as but not limited to Dulbecco's Modified Eagle's Medium (DMEM), Endothelial differentiation medium (EDM), Minimal Essential Medium (MEM), Basal Medium Eagle (BME), RPMI 1640, F-10, F-12, α-Minimal Essential Medium (α-MEM), Glasgow's Minimal Essential Medium (G-MEM), and Iscove's Modified Dulbecco's Medium. Additionally, the conditioned medium to be used may be any basic culture medium suitable for animal cell growth, and non-limiting examples of the medium may include Minimal Essential Medium (MEM), Dulbecco modified Eagle Medium (DMEM), Roswell Park Memorial Institute Medium (RPMI), and Keratinocyte Medium (KM), KBM (Keratinocyte Basal medium), EpiLife KM (Keratinocyte-EpiLife medium) etc. In some examples, conditioned medium comprises a cell culture medium as described herein, such as a culture medium comprising DMEM (Dulbecco's modified eagle medium), F12 (Ham's F12 Medium), M171 (Medium 171) and FBS (Fetal Bovine Serum).

In another aspect, the present invention provides a conditioned medium derived from mesenchymal stem cells of the umbilical cord obtained or obtainable by the method according to the method of the present invention. In a related aspect, the present invention provides a composition comprising the conditioned medium of the present invention. In some embodiments, the composition as described herein is a pharmaceutical or cosmetic composition for inducing, stimulating and/or promoting healing of a wound and/or preventing formation and/or recurrence of a wound, and/or treating rosacea, psoriasis, eczema, dermatitis, topical steroid withdrawal syndrome, epidermolysis bullosa. The composition may be prepared in any formulation conventionally manufactured in the art, and usually comprises conditioned medium of the present invention together with a cosmetically or pharmaceutically suitable acceptable carrier/recipient.

In some examples, the present invention provides a composition comprising the conditioned medium derived from cultivation of mesenchymal stem cells of the umbilical cord that is formulated to be used for skin care or skin treatment. Examples of such formulations will be described in more detail in the following.

In examples of such a composition, the composition may further comprise an anti-inflammatory compound. Any suitable anti-inflammatory compound can be used in such a composition. The anti-inflammatory compound may be of natural origin or a chemically synthesized small molecule drug. In illustrative examples, the anti-inflammatory compound may be curcumin, sulfacetamide, a non-steroidal anti-inflammatory compound, for example capsaicin or diclofenac, niacinamide (vitamin B3), sea buckthorn oil, calendula, chamomile (botanical) extract or extracts of Centella asiatica to mention only a few. The anti-inflammatory properties of curcumin are reviewed in Peng et al, “Anti-Inflammatory Effects of Curcumin in Inflammatory Diseases: Status, Limitations and Countermeasures”, Drug Design, Development and Therapy 2021:15 4503-4525, for example. Calendula is a natural oil extracted from marigold flowers native to the Mediterranean region. This sweet juice contains flavonoids with antiseptic properties, and carries anti-inflammatory abilities that help heal wounds and calm eczema. Chamomile (botanical) extract is the powdered essence of German (also known as Hungarian, or Blue) Chamomile, known scientifically as Matricaria recutita and has been used both in traditional remedies for wound healing as well as hamomile Botanical Extract is the powdered essence of German (also known as Hungarian, or Blue) Chamomile, known scientifically as Matricaria recutita. In addition of being used in traditional remedies for wound healing, chamomile extract is regularly included in skincare products that address irritation and inflammation. Anti-inflammatory properties of extracts of Centella asiatica are discussed in Gohil et al., “Pharmacological Review on Centella asiatica: A Potential Herbal Cure-all”, Indian J Pharm Sci. 2010 September-October; 72(5): 546-556.

The composition may further comprise a humectant. The term humectant is used here in its regular meaning in the field of skin care and refers to components that attract moisture to the skin. Any suitable humectant can be used in a respective composition described herein. Illustrative examples of humectants include, but are not limited to glycerine, propylene glycol, triethylene glycol, tripropylene glycol, sorbitol, hexylene glycol, butylene glycol, urea, collagen, butylene glycol, aloe vera gel and mixtures thereof. In some examples of compositions described herein the humectant comprises glycerine, propylene glycol or both glycerine and propylene glycol (see also below).

A composition comprising conditioned medium, an anti-inflammatory compound and a humectant may comprise the conditioned medium derived from cultivation of mesenchymal stem cells of the umbilical cord in an amount of about 5 to 40% (v/v) of the total volume of the composition, the humectant in amount of about 10 to 30% (v/v) of the total volume of the composition, and the anti-inflammatory compound in an amount of about 1.0 to about 3.0% (v/v) of the composition or in an amount of 0.5 to 5.0% (w/w) of the total weight of the composition. In other examples, such a composition may comprise the conditioned medium derived from cultivation of mesenchymal stem cells of the umbilical cord in an amount of about 10 to 30% (v/v) of the total volume of the composition, the humectant in an amount of about 15 to 25% (v/v) of the total volume of the composition, and the anti-inflammatory compound in an amount of about 1.0 to about 3.0% (v/v) of the composition or in an amount of 0.75 to 3.0% (w/w) of the total weight of the composition.

Alternatively, a composition comprising conditioned medium, an anti-inflammatory compound and a humectant may comprise the conditioned medium derived from cultivation of mesenchymal stem cells of the umbilical cord in an amount of about 5 to 40% (w/w) of the total weight of the composition, the humectant in amount of about 10 to 30% (w/w) of the total weight of the composition, and the anti-inflammatory compound in an amount of about 0.5 to 5.0% (w/w) of the total weight of the composition. For the sake of clarity, it is noted here that volume percentages (% v/v) are preferably used for defining the content of a component in the composition, if the component is typically present in liquid form at room temperature, while weight percentages (% w/w) are preferably used, if the component is typically present in solid form at room temperature.

In illustrative examples of such a composition, the composition comprises
conditioned medium derived from cultivation of mesenchymal stem cells of the umbilical cord,
glycerine, propylene glycol or both glycerine and propylene glycol, and
curcumin.

A composition as described herein may further comprise a solvent/carrier. Any cosmetically acceptable solvent/carrier may be used. In illustrative examples, the solvent/carrier may be water or an aqueous buffer solution.

A composition as described herein may further comprises a preservative. A preservative as used herein is used in its regular meaning to refer to a natural or synthetic ingredient that protects the integrity of a composition (formulation). Preservatives may avoid growth of bacteria or other microorganism. Illustrative example of suitable preservatives include, but are not limited to 1,2-hexanediol, benzyl alcohol, benzyl benzoate, benzalkonium chloride, citric acid, a paraben, a formaldehyde-releasing preservative (e.g. DMDM hydantoin diazolydinal urea or imidiazoldinal urea), anisic acid, salicylic acid, sodium salicylate, chlorphenism, pr stearalkonium chloride to name only a few.

In illustrative examples such a composition as described herein may comprise
water in an amount of about 50 to about 65% (v/v), preferably about 55 to about 58% (v/v), most preferably in an amount of 57% (v/v) of the total volume of the composition
glycerin in an amount of about 10 to about 35% (v/v), preferably about 15 to about 25% (v/v), most preferably about 20% (v/v) of the total amount of the composition
conditioned medium in an amount of about 10 to about 35% (v/v), preferably about 15 to about 25% (v/v), most preferably 20% (v/v) of the total amount of the composition,
1,2-hexanediol in an amount of about 1 to about 55% (v/v), preferably about 1.5 to about 3.5% (v/v), most preferably 2% (v/v) of the total amount of the composition,
curcumin in an amount of about 0.5 to about 5.0% (w/w), preferably of about 0.75 to about 2.5% (w/w), most preferably 1.0% (w/w) of the total weight of the composition.

In other illustrative examples such a composition as described herein may comprise
water in an amount of about 40 to about 65% (w/w), preferably about 45 to about 55% (w/w), most preferably in an amount of about 52% (w/w) of the total weight of the composition
the conditioned medium in an amount of about the umbilical cord in an amount of about 5 to about 40% (w/w) of the total weight of the composition, preferably in an amount of about 10% (w/w) to about 30% (w/w) or about 12.5 to about 25% (w/w), most preferably in a amount of about 17.5 to 20.0% (w/w) %,
glycerin and/or propylene glycol in amount of about 10 to 30% (w/w) of the total weight of the composition, preferably in an amount of about 10% (w/w) to about 30% (w/w) or about 12.5 to about 25% (w/w), most preferably in an amount of about 17.5 to 20.0% (w/w) of the total weight of the composition, and
curcumin in about 0.5 to 5.0% (w/w) of the total weight of the composition, preferably about 0.75 to about 2.5% (w/w) or 0.8 to about 1.5% (w/w), most preferably about 0.9 to 1.0% (w/w) of the total weight of the composition.

A composition as described here may further comprise one or more of a gelling agent, a buffering agent and an emollient. Any suitable gelling agent can be used in such a composition. Illustrative examples of gelling agents include but are not limited to wherein the gelling agent is selected from the group consisting of a poly(acrylic acid), pectin, starch, an alginate such as sodium alginate, gelatin, a cellulose derivative (e.g. hypromellose, or methylcellulose) and polyvinyl alcohol clay.

The “buffering agent” is used herein in its regular meaning to stabilize or adjust the pH of a cosmetic product. Likewise, the term “emollient” is used in its regular meaning in cosmetic formulating to refer to compounds that are able to soften skin and are incorporated into a cosmetic product to improve the feel of skin. Also any suitable buffering agent can be used in compositions as described herein. Illustrative examples of buffering agents include, but are not limited to, sodium phosphate salt, calcium carbonate, sodium bicarbonate, citric acid, triethanolamine and a Good's buffer. Examples of suitable Good's buffer are HEPES, Tris, BisTris, Glycylglycine, MOPS (3-(N-morpholino) propanesulfonic acid), and Tricine, to name only a few. Likewise, any suitable emollient can be used in compositions as described herein with illustrative examples of suitable emollients being Olive Oil PEG-7 Esters, triglycerides, lanolin, polyhydric alcohols, and esters of fatty acids (e.g. isopropyl myristate, dioctyl sebecate and dioctyl maleate).

Further illustrative examples of compositions of the invention are shown in the following Tables.

TABLE 1
Exemplary composition of the invention
that is formulated as a lotion

| Ingredient (NCI name) | Amount | Purpose |
|---|---|---|
| Aqua (water) | 57.00%, (v/v) | Solvent/Carrier |
| Glycerin (glycerol) | 20.00%, (v/v) | Humectant |
| Conditioned Medium | 20.00% (v/v) | Active ingredient for skin |
| | | care and wound healing |
| 1,2-Hexanediol | 2.00%, (v/V) | Preservative |
| Curcumin | 1.00%, (w/w) | Anti-inflammatory agent |

It is noted here that the amount of curcumin is given in weight percent of the total weight of the formulation since curcumin is a solid (powder) while the amount of the other components is given in volume percent of the total volume of the formulation since these components are in a liquid state at room temperature. If instead, the anti-inflammatory agent is, for example, Calendula (an oil), its amount in the composition is typically defined in volume percent of the total volume of the formulation.

A lotion as described in Table 1 can, for example, be advantageously used as a daily skin conditioner for use by a patient at home, for example, to prevent formation and/or recurrence of a wound. For this purpose, a patient such as a diabetic patient can topically administering the lotion to the skin of the foot or ankles to improve the condition of the diabetic skin and thereby avoiding formation of a diabetic wound such as a foot ulcer.

TABLE 2
Exemplary composition of the invention that is formulated as a gel

| Ingredients | Amount % (w/w) | Purpose |
|---|---|---|
| Aqua | 51.9954 | Solvent/carrier |
| Glycerin | 18.2440 | Humectant |
| Conditioned Medium | 18.2440 | Active ingredient for skin |
| | | care and wound healing |
| Propylene Glycol | 5.0000 | Humectant |
| Olive Oil PEG-7 Esters | 3.0000 | Emollient |
| 1,2-Hexanediol | 1.8244 | Preservative |
| Curcumin | 0.9122 | Anti-inflammatory agent |
| | | (anti-oxidant) |
| Carbomer | 0.5000 | Gelling agent |
| Tromethamine (Tris) | 0.2800 | Buffer |
| Total | 100.0000 | |

A gel as described in Table 2 can, for example, be advantageously used for inducing, stimulating and/or promoting healing of a wound or of a damaged skin area, wherein the method comprises topically administering conditioned medium derived from cultivation of mesenchymal stem cells of the umbilical cord to the skin of a patient that surrounds the wound such a pressure ulcer or a diabetic foot ulcer.

The compositions of the invention may also contain further additives and adjuvants which are conventional in the cosmetic, pharmaceutical or dermatological field such as fragrances, fillers, bactericides, odor absorbers and dyestuffs or colorants. Typically, the composition may be used via topical/dermal application by directly applying it on the skin. Preferably, a composition comprising conditioned medium of the present invention is a cosmetic composition. In some embodiments, the composition is adapted for topical application. A composition adapted for topical application may be in liquid or viscous form. Examples thereof include a gel, an ointment, a cream, and a lotion and the like. The preparation of such compositions is within the knowledge of the person skilled in the art and described in Gennaro, A. L. and Gennaro, A. R. (2000) Remington: The Science and Practice of Pharmacy, 20th Ed., Lippincott Williams & Wilkins, Philadelphia, PA, for example. In this context, a composition comprising conditioned medium of the present invention for topical application is to be understood as a formulation that is able to be absorbed by the skin in a targeted manner, particularly wherein the active ingredients of the conditioned medium of the present invention or the composition comprising the conditioned medium of the present invention are able to penetrate the layers of the epidermis and dermis, but not to be absorbed into the systemic circulation. In some examples, the conditioned medium of the present invention or the composition comprising the conditioned medium of the present invention crosses the surface of the skin by trans-epidermal diffusion or by transdermal diffusion.

In a further aspect, the present invention provides the use of conditioned medium derived from cultivation of mesenchymal stem cells of the umbilical cord for medium for inducing, stimulating and/or promoting healing of a wound or damaged skin area and/or preventing formation and/or recurrence of a wound or damaged skin area, and/or treating rosacea, psoriasis, eczema, dermatitis, topical steroid withdrawal syndrome, epidermolysis bullosa. In yet a further aspect, the present invention provides the use of conditioned medium derived from cultivation of mesenchymal stem cells of the umbilical cord for the manufacture of a medicament for inducing, stimulating and/or promoting healing of a wound or damaged skin area and/or preventing formation and/or recurrence of a wound or damaged skin area, and/or treating rosacea, psoriasis, eczema, dermatitis, topical steroid withdrawal syndrome, epidermolysis bullosa. Accordingly, a method of treating a subject with conditioned medium derived from cultivation of mesenchymal stem cells of the umbilical cord is described. This method comprises administering to the skin of a subject a (therapeutically) effective amount of the conditioned medium of the present invention. A treatment course can include repeated administration of such an effective amount over any desired period of time. As described herein, a composition/formulation that contain conditioned medium may, for example, be applied once daily or twice or three times a week, over any desired period of time. See Example 3, in which formulations containing conditioned medium as described herein were applied for up to 16 weeks, twice to three times a week, to achieve closure of chronic diabetic foot ulcers. The person skilled in the art is aware of the fact that the period of time over which formulations of the invention are applied depend on the specific situation of a patient (age, health status, size of the wound etc.) but that treatment can be carried out until the desired effect has been achieved. As also illustrated in the Example Section (see again Example 3), an “effective amount of the conditioned medium” may be any amount of a formulation that contains conditioned medium (for example in an amount of about 20% (w/w) based on the total weight of the formulation) and that is applied per treatment to the skin of a subject to cover the desired skin area.

In principle, any subject is suitable for being treated by means of conditioned medium of the present invention. Finally, the invention also provides method of treating a non-human mammal or a human subject, the method comprising topically treating the skin of a subject surrounding the wound or damaged skin area or the intact skin of a subject being at risk of developing a wound or damaged skin area with conditioned medium derived from cultivation of mesenchymal stem cells of the umbilical cord.

The invention will be further illustrated by the following non-limiting Experimental Examples.

Sequences as used herein are depicted in below Table 3.

TABLE 3
Amino acid sequences as described herein.

| SEQ ID NO. | What | Sequence |
|---|---|---|
| 1 | PDGFA | MRTLACLLLLGCGYLAHVLAEEAEIPREVIERLARSQIHSIR |
| | (PDGF-AA) | DLQRLLEIDSVGSEDSLDTSLRAHGVHATKHVPEKRPLPIR |
| | human | RKRSIEEAVPAVCKTRTVIYEIPRSQVDPTSANFLIWPPCVE |
| | Uniprot no: | VKRCTGCCNTSSVKCQPSRVHHRSVKVAKVEYVRKKPKL |
| | P04085, | KEVQVRLEEHLECACATTSLNPDYREEDTGRPRESGKKRK |
| | of Feb. | RKRLKPT |
| | 10,2009 | |
| 2 | PDGFB | MNRCWALFLSLCCYLRLVSAEGDPIPEELYEMLSDHSIRSF |
| | human | DDLQRLLHGDPGEEDGAELDLNMTRSHSGGELESLARGR |
| | Uniprot no: | RSLGSLTIAEPAMIAECKTRTEVFEISRRLIDRTNANFLVWP |
| | P01127 | PCVEVQRCSGCCNNRNVQCRPTQVQLRPVQVRKIEIVRKK |
| | of Feb. | PIFKKATVTLEDHLACKCETVAAARPVTRSPGGSQEQRAK |
| | 06, 2013 | TPQTRVTIRTVRVRRPPKGKHRKFKHTHDKTALKETLGA |
| 3 | TGFB1 (TGF- | MPPSGLRLLLLLLPLLWLLVLTPGRPAAGLSTCKTIDMELVK |
| | (1) | RKRIEAIRGQILSKLRLASPPSQGEVPPGPLPEAVLALYNST |
| | human | RDRVAGESAEPEPEPEADYYAKEVTRVLMVETHNEIYDKF |
| | Uniprot no: | KQSTHSIYMFFNTSELREAVPEPVLLSRAELRLLRLKLKVE |
| | P01137 | QHVELYQKYSNNSWRYLSNRLLAPSDSPEWLSFDVTGVV |
| | of Oct. | RQWLSRGGEIEGFRLSAHCSCDSRDNTLQVDINGFTTGRR |
| | 13, 2009 | GDLATIHGMNRPFLLLMATPLERAQHLQSSRHRRALDTNY |
| | | CFSSTEKNCCVRQLYIDFRKDLGWKWIHEPKGYHANFCLG |
| | | PCPYIWSLDTQYSKVLALYNQHNPGASAAPCCVPQALEPL |
| | | PIVYYVGRKPKVEQLSNMIVRSCKCS |
| 4 | IGF1 | MGKISSLPTQLFKCCFCDFLKVKMHTMSSSHLFYLALCLLT |
| | human | FTSSATAGPETLCGAELVDALQFVCGDRGFYFNKPTGYGS |
| | Uniprot no: | SSRRAPQTGIVDECCFRSCDLRRLEMYCAPLKPAKSARSV |
| | P05019 | RAQRHTDMPKTQKYQPPSTNKNTKSQRRKGWPKTHPGG |
| | of Jun. 26, | EQKEGTEASLQIRGKKKEQRREIGSRNAECRGKKGK |
| | 2013 | |
| 5 | FGF2 (bFGF) | MGDRGRGRALPGGRLGGRGRGRAPERVGGRGRGRGTA |
| | human | APRAAPAARGSRPGPAGTMAAGSITTLPALPEDGGSGAFP |
| | Uniprot no: | PGHFKDPKRLYCKNGGFFLRIHPDGRVDGVREKSDPHIKL |
| | P09038 | QLQAEERGVVSIKGVCANRYLAMKEDGRLLASKCVTDECF |
| | Jul. 28, 2009 | FFERLESNNYNTYRSRKYTSWYVALKRTGQYKLGSKTGP |
| | | GQKAILFLPMSAKS |
| 6 | FGF7 | MHKWILTWILPTLLYRSCFHIICLVGTISLACNDMTPEQMAT |
| | (KGF) | NVNCSSPERHTRSYDYMEGGDIRVRRLFCRTQWYLRIDKR |
| | Human | GKVKGTQEMKNNYNIMEIRTVAVGIVAIKGVESEFYLAMNK |
| | Uniprot no: | EGKLYAKKECNEDCNFKELILENHYNTYASAKWTHNGGEM |
| | P21781 | FVALNQKGIPVRGKKTKKEQKTAHFLPMAIT |
| | Jul. 9, 2014 | |
| 7 | VEGFA | MNFLLSWVHWSLALLLYLHHAKWSQAAPMAEGGGQNHH |
| | human | EVVKFMDVYQRSYCHPIETLVDIFQEYPDEIEYIFKPSCVPL |
| | Uniprot no: | MRCGGCCNDEGLECVPTEESNITMQIMRIKPHQGQHIGEM |
| | P15692 | SFLQHNKCECRPKKDRARQEKKSVRGKGKGQKRKRKKSR |
| | Apr. 16, 2014 | YKSWSVYVGARCCLMPWSLPGPHPCGPCSERRKHLFVQ |
| | | DPQTCKCSCKNTDSRCKARQLELNERTCRCDKPRR |
| 8 | VEGFB | MSPLLRRLLLAALLQLAPAQAPVSQPDAPGHQRKVVSWID |
| | human | VYTRATCQPREVVVPLTVELMGTVAKQLVPSCVTVQRCG |
| | Uniprot no: | GCCPDDGLECVPTGQHQVRMQILMIRYPSSQLGEMSLEE |
| | P49765 | HSQCECRPKKKDSAVKPDRAATPHHRPQPRSVPGWDSA |
| | Nov. | PGAPSPADITHPTPAPGPSAHAAPSTTSALTPGPAAAAADA |
| | 16, 2001 | AASSVAKGGA |
| 9 | IL10 | MHSSALLCCLVLLTGVRASPGQGTQSENSCTHFPGNLPN |
| | Human | MLRDLRDAFSRVKTFFQMKDQLDNLLLKESLLEDFKGYLG |
| | Uniprot no: | CQALSEMIQFYLEEVMPQAENQDPDIKAHVNSLGENLKTL |
| | P22301 | RLRLRRCHRFLPCENKSKAVEQVKNAFNKLQEKGIYKAMS |
| | Aug. 1, | EFDIFINYIEAYMTMKIRN |
| | 1991 | |
| 10 | ANGP1 | MTVFLSFAFLAAILTHIGCSNQRRSPENSGRRYNRIQHGQC |
| | (ANG-1) | AYTFILPEHDGNCRESTTDQYNTNALQRDAPHVEPDFSSQ |
| | human | KLQHLEHVMENYTQWLQKLENYIVENMKSEMAQIQQNAV |
| | Uniprot no: | QNHTATMLEIGTSLLSQTAEQTRKLTDVETQVLNQTSRLEI |
| | Q15389 | QLLENSLSTYKLEKQLLQQTNEILKIHEKNSLLEHKILEMEG |
| | of Apr. 3, | KHKEELDTLKEEKENLQGLVTRQTYIIQELEKQLNRATTNN |
| | 2013 | SVLQKQQLELMDTVHNLVNLCTKEGVLLKGGKREEEKPFR |
| | | DCADVYQAGFNKSGIYTIYINNMPEPKKVFCNMDVNGGGW |
| | | TVIQHREDGSLDFQRGWKEYKMGFGNPSGEYWLGNEFIF |
| | | AITSQRQYMLRIELMDWEGNRAYSQYDRFHIGNEKQNYRL |
| | | YLKGHTGTAGKQSSLILHGADFSTKDADNDNCMCKCALML |
| | | TGGWWFDACGPSNLNGMFYTAGQNHGKLNGIKWHYFKG |
| | | PSYSLRSTTMMIRPLDF |
| 11 | HGF | MWVTKLLPALLLQHVLLHLLLLPIAIPYAEGQRKRRNTIHEF |
| | human | KKSAKTTLIKIDPALKIKTKKVNTADQCANRCTRNKGLPFTC |
| | Uniprot no: | KAFVFDKARKQCLWFPFNSMSSGVKKEFGHEFDLYENKD |
| | P14210 | YIRNCIIGKGRSYKGTVSITKSGIKCQPWSSMIPHEHSFLPS |
| | of Dec. | SYRGKDLQENYCRNPRGEEGGPWCFTSNPEVRYEVCDIP |
| | 16, 2008 | QCSEVECMTCNGESYRGLMDHTESGKICQRWDHQTPHR |
| | | HKFLPERYPDKGFDDNYCRNPDGQPRPWCYTLDPHTRW |
| | | EYCAIKTCADNTMNDTDVPLETTECIQGQGEGYRGTVNTI |
| | | WNGIPCQRWDSQYPHEHDMTPENFKCKDLRENYCRNPD |
| | | GSESPWCFTTDPNIRVGYCSQIPNCDMSHGQDCYRGNGK |
| | | NYMGNLSQTRSGLTCSMWDKNMEDLHRHIFWEPDASKLN |
| | | ENYCRNPDDDAHGPWCYTGNPLIPWDYCPISRCEGDTTP |
| | | TIVNLDHPVISCAKTKQLRVVNGIPTRTNIGWMVSLRYRNK |
| | | HICGGSLIKESWVLTARQCFPSRDLKDYEAWLGIHDVHGR |
| | | GDEKCKQVLNVSQLVYGPEGSDLVLMKLARPAVLDDFVST |
| | | IDLPNYGCTIPEKTSCSVYGWGYTGLINYDGLLRVAHLYIM |
| | | GNEKCSQHHRGKVTLNESEICAGAEKIGSGPCEGDYGGPL |
| | | VCEQHKMRMVLGVIVPGRGCAIPNRPGIFVRVAYYAKWIH |
| | | KIILTYKVPQS |

### EXPERIMENTAL EXAMPLES

### Example 1: Isolation and Cultivation of Mesenchymal Stem Cells of the Umbilical Cord (CLMSCs)

### Culture Medium Preparation

a. To make 500 ml PTT-6 (culture/growth medium) add the following in the order listed:
i. DMEM, 250 ml
ii. M171 118 ml
iii. DMEMF12 118 ml
iv. FBS 12.5 ml (final concentration of 2.5%)
v. EGF 1 ml (final concentration of 10 ng/ml)
vi. Insulin 0.175 ml (final concentration of 5 μg/ml)

The above-mentioned volumes of components i. to vi result in a final volume of 499.675 ml culture medium. If no further components are added to the culture medium, the remaining 0.325 ml (to add up to a volume of 500 ml) can, for example, be any of components i. to iv, that means either DMEM, M171, DMEM/F12 or FBS. Alternatively, the concentration of the stock solution of EGF or Insulin can of course be adjusted such that the total volume of the culture medium is 500 ml. Alternatively, a stock solution of an antibiotic such as Penicillin-Streptomycin-Amphotericin can be added to result in a final volume of 500 ml. It is also possible to add to the culture medium a volume of 0.325 ml of one or more of the following supplements: adenine, hydrocortisone, 3,3′,5-Triiodo-Fthyronine sodium salt (T3), thereby reaching a total volume of 500 ml culture medium.
vii. Label the bottle “PTT-6” with date medium was prepared, initial of the operator, and the phrase “expires on” followed by the expiration date. Expiration date is the earliest expiration date of any of the component or 1 month from the preparation date, whichever comes first.
b. To make the rinse medium (Hank's Buffered Salt Solution (HBSS) without Calcium or Magnesium and with 5% FBS), add 2.5 ml FBS to 47.5 ml of HBSS in a 50 ml centrifuge tube. Label the tube “Rinse Medium” with operator initials and date the medium is made.
c. All medium are tested for sterility using Bactec Fytic/10 Dickinson & Company) and Bactec Pluc+Aerobic/F (Becton Dickinson & Company). Inject 20 ml of prepared medium into each bottle.

### Collection and Isolation of Red Deer (RD) Umbilical Cord Tissue

Red deer (RD) umbilical cords are collected under specific handling instructions from a farm in New Zealand that rears the animal for collection of horn velvet. RD-CLMSC are isolated from umbilical cords according to the protocol described in International Patent Application WO 2006/019357 A1.

In brief, the umbilical cords of red deer are rinsed clean and immediately transferred into a 500 ml sterile glass bottle containing culture transport medium (L-15 medium supplemented with 50 IU/ml penicillin, 50 μg/ml streptomycin, 250 μg/ml fungizone, 50 μg/ml gentamicin; all reagents purchased from Invitrogen) prior to transport to the laboratory. In the laboratory, stem cell extraction is conducted in a laminar flow hood under sterile conditions. The umbilical cords are first transferred to a sterile stainless steel tray. All remaining blood in the cord vessels is removed by multiple syringing washes using warm phosphate-buffered saline (PBS) supplemented with 5 IU/ml heparin (from Sigma). Plain PBS without heparin is used in the final washes. The umbilical cord tissue specimen is then cut into pieces 2 cm in length and transferred into 10 cm diameter cell culture dishes, where further washing and disinfection is performed with 70% ethanol followed by multiple washes using PBS containing an antibiotic mixture (50 IU/ml penicillin, 50 μg/ml streptomycin, 250 μg/ml fungizone, 50 μg/ml gentamicin; all purchased from Invitrogen) until the solution becomes clear.

### Isolation and Cultivation of RD-CLMSC

Dissection of umbilical cord tissue is first performed to separate the umbilical cord amniotic membrane from Wharton's jelly (i.e. the matrix of umbilical cord) and other internal components. The isolated amniotic membrane is then cut into small pieces (0.5 cm×0.5 cm) for cell isolation. Explant is performed by placing the pieces of umbilical cord amniotic membrane on tissue culture dishes at different cell culture conditions for isolation of mesenchymal stem cells.

For mesenchymal cell isolation/cultivation, the explants are submerged in PTT-6 medium (CellResearch Corp, Singapore) and maintained in a CO₂ cell culture incubator at 37° C. The medium is changed every 2 or 3 days. Cell outgrowth is monitored under light microscopy. At a confluence of about 70%, cells are trypsinized (0.05% trypsin/0.02% EDTA) for further expansion or for cryo-preservation. H-CLMSC (#CLMC43) was also obtained from the CRC tissue bank.

### Example 2: Conditioned Medium Derived from Red Deer Cord Lining Mesenchymal Stem Cells (RD-CLMSCs)

Cryovials containing RD-CLMSC are retrieved from storage and quick thawed in a 37° C. water bath. FSF was given to CRC (CellResearch Corporation, Singapore) as a gift from the Stem Cell and Wound Healing Research Group, Department of Surgery, Yong Loo Lin School of Medicine, National University of Singapore.

PTT-6 medium (CellResearch Corporation, Singapore) is used for culturing RD-CLMSCs at 37° C. with 5% CO₂. The cell culture medium is changed every 2 to 3 days. At 80% to 100% confluency, the cell culture medium is removed and cells may optionally be rinsed in PBS before cultured in DMEM basal medium, wherein the medium optionally comprises a water-soluble antioxidant but no growth factors or serum. Culture dishes are incubated for further 48 hours and spent medium is collected into centrifuge tubes for centrifugation at 1800 rpm for 10 minutes. The supernatant is collected as conditioned medium (CM) into labelled tubes. Conditioned media are kept at −80° C. until use.

### Example 3: Evaluation of Conditioned Medium in Healing Persistent Chronic Diabetic Foot Ulcer (DFU) Wounds

### Experimental Set Up

The conditioned medium is derived from red deer umbilical cord lining stem cell conditioned medium as outlined in Examples 1 and 2 above and was formulated either as a liquid solution or a gel as described in Table 1 above, containing about 20% (w/w) conditioned medium.

Patients with a chronic diabetic foot ulcer (duration of >3 months) were recruited to this case series via hospital DFU registry. In all cases, DFU persisted despite a standard of care, including regular debridement by podiatrists, topical dressings and offloading shoe wear. Patients with active infection were excluded from the case series. Conditioned medium was applied to clean, debrided intact skin surrounding the wound (ISSW), either via the liquid solution soaked in a gauze, or via the gel formulation as described in Table 1 above (that contains about 20% (w/w) conditioned media) that was applied directly to the ISSW. After 5 minutes the gauze or excess gel is removed and the wound dressed as per routine care. Treatment is either twice or three times weekly.

21 patients were recruited: 15 males and 6 females. The average age of patients was 56.7 years (range 40-72 years) with mean HbA1c of 7.9% (SD=1.99). DFUs undergoing treatment were in a range of locations on the foot, with the majority being plantar ulcers. No significant systematic complications such as allergic reactions or sepsis were observed during the duration of application.

### Methodology

The wounds of the patients were treated as follows:
i) The wound is debrided and cleansed as per routine;
ii) 5 ml of conditioned medium formulation is sauced in gauze;
iii) gauze is applied to intact skin surrounding wound (ISSW) for 5 minutes or alternatively the gel is applied to the ISSW;
iv) conditioned medium is not administered directly on any open skin or wound;
v) gauze or excess gel is removed; and
vi) prescribed topical dressings as per previous treatment is presumed

### Results

At the time of result collection 12 of 21 patients demonstrated total DFU closure, with ulcer closure appearing to be faster in those patients treated with the gel formulation. Of the remaining patients, 8 (5 of which are still in treatment) experienced a significant reduction in their wound size. 1 patient saw no improvement.

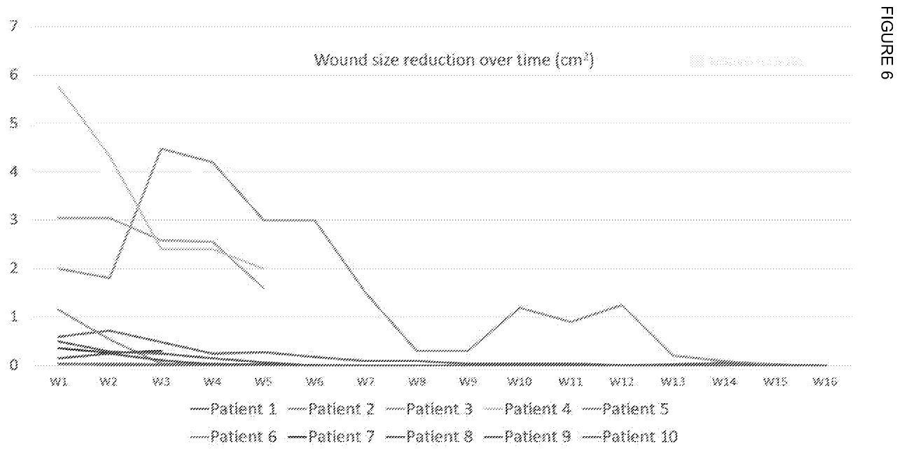

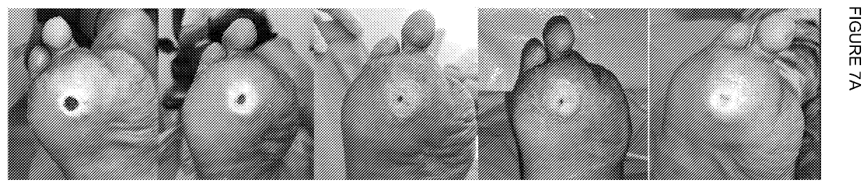

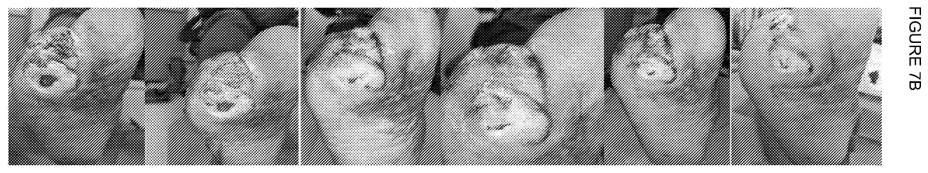

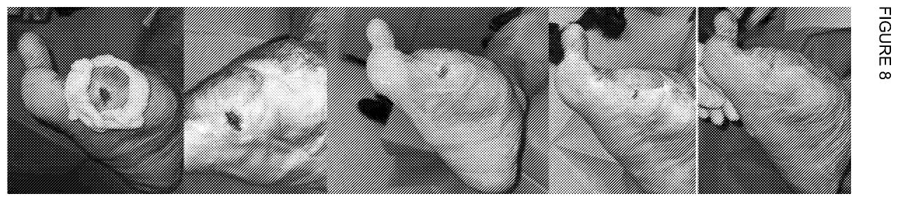

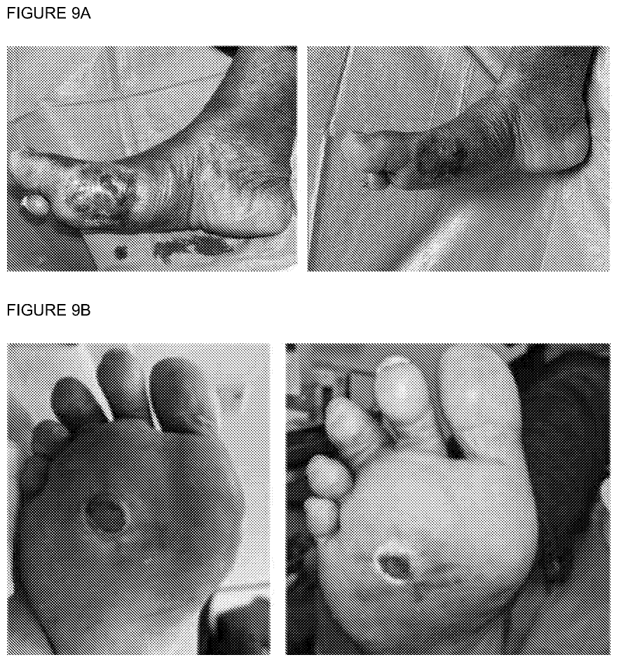

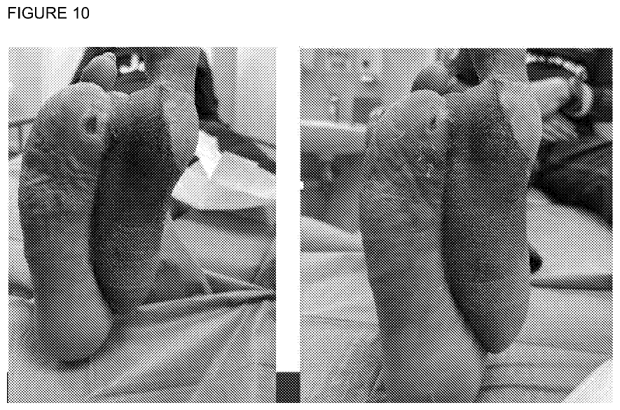

Reduction in wound size in the subpopulation of the 10 patients that were treated with a gauze soaked with the conditioned medium is shown in FIG. 6 and exemplary photographs of patients of healed wounds are shown in FIG. 7-10. In this patient group were 8 male and 2 female patients. The mean age of these patients were 54.7 (SD=9.4) years, with mean HbA1c of 8.2% (SD=2.5). 8 (80%) patients had at least 1 palpable distal foot pulse. 6 patients (60%) experienced wound healing during the treatment period, 3 patients (30%) demonstrated reduced wound sizes, and 1 (10%) patient had stable wound size. Of those who healed, patients who applied the conditioned medium twice a week (n=2) healed in 16 weeks, and patients that applied conditioned medium three times a week (n=4) healed in 4 to 7 weeks. In this context, patients demonstrated a significant reduction in their wound size and wound closure. Particularly, as exemplarily shown in FIG. 7-10, administration of the conditioned medium to the intact skin surrounding the wound (ISSW) promoted healing of chronic persistent wounds of DFUs, thereby regenerating the skin and restoring healthy skin. No patient experienced any side effects and no systemic or local safety concerns have been observed to the treatment with conditioned medium.

In this context, it is referred to the result of a Phase 3 pivotal, single-blind clinical trial in which DFU were treated with Dermagraft. In this clinical trial stratification was by ulcer size (≥1 to ≤2 cm² and >2 to ≤20 cm²) and the key inclusion criteria were; Chronic DFU, >6 weeks wound duration; ulcer size 1-20 cm², extension through dermis permitted (without exposure to muscle, tendon, bone, or joint capsule). Dermagraft was applied at day 0 followed by up to 7 additional applications at weekly intervals together with standard care, the trial included 130 patients (n=130). The outcome of this trial was as follows: Incidence of complete wound closure at 12 weeks was 30.0% (39 of 130 patients) versus 18.3% (21 of 115 patients) in the control group (P=0.023). While the results of the present study with 21 patients and no control group are not directly comparable with the results of this clinical trial with Dermagraft, the wound closure rate of about 57% (12 of 21 patients demonstrating total DFU closure, see above), indicates that treatment of chronic DFU with conditioned medium as described herein should be at least as effective or even more effective than treatment with Dermagraft as a tissue-engineered, living human dermal substitute approved by the US FDA for treatment of DFU.

Thus, the results of the present application show that administering condition medium of mesenchymal stem cells of the umbilical cord tissue such as the cord lining, onto the skin surrounding a wound such as a chronic diabetic foot ulcer, leads to efficient wound closure, thereby providing a new, inexpensive, and easy to apply approach to treat such difficult to treat wounds.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group. Further embodiments of the invention will become apparent from the following claims.

What is claimed is:

1. A method of inducing, stimulating and/or promoting healing of a wound or of a damaged skin area, wherein the method comprises topically administering to the skin of a subject surrounding the wound or the damaged skin area conditioned medium derived from cultivation of mesenchymal stem cells of the umbilical cord.

2. A method of preventing formation and/or recurrence of a wound or of a damaged skin area in a subject being at risk of developing a wound or a damaged skin area, wherein the method comprises topically administering to the skin of a subject conditioned medium derived from cultivation of mesenchymal stem cells of the umbilical cord.

3. A method of treating rosacea, psoriasis, eczema, dermatitis, topical steroid withdrawal syndrome, epidermolysis bullosa, or skin damage caused by fragile skin, wherein the method comprises topically administering to the skin of a subject conditioned medium derived from cultivation of mesenchymal stem cells of the umbilical cord.

4. The method of claim 1, wherein the method does not comprise administering the conditioned medium onto the wound or the damaged skin area.

5. The method of any of claims 1-4, wherein the conditioned medium is administered in the form of a gel, a cream or a liquid.

6. The method of any of claims 1-5, wherein the skin to be treated is the intact skin.

7. The method of claims 1-6, wherein the skin to be treated is diabetic skin or thin (weak) skin.

8. The method of any of claims 1-7, wherein the wound or damaged skin area is a chronic wound or a chronically damaged skin.

9. The method of any of claims 1-8, wherein the damaged skin area is a wound.

10. The method of any of claims 1-9, wherein the wound or damaged skin area is selected from the group consisting of diabetic foot ulcer (DFU), pressure ulcer, a wound due to chronic limb ischemia, a wound due to chronic limb-threatening limb ischemia, venous leg ulcer, a surgical wound, a stab wound, a burn wound, and a bite wound.

11. The method of any of the preceding claims, wherein the conditioned medium is derived from cultivation of a mesenchymal stem cell population of the amniotic membrane of the umbilical cord, from cultivation of a mesenchymal stem population of the Wharton's jelly of the umbilical cord (WJ), from cultivation of a perivascular (PV) mesenchymal stem cell population, or from cultivation of a mixed mesenchymal stem cell population of the umbilical cord (MC).

12. The method of any of the preceding claims, wherein the conditioned medium is obtained or obtainable by cultivation of mesenchymal stem cells of the umbilical cord in a culture medium comprising DMEM (Dulbecco's modified eagle medium), F12 (Ham's F12 Medium), M171 (Medium 171) and FBS (Fetal Bovine Serum).

13. The method of claim 12, wherein the culture medium comprises DMEM in a final concentration of about 55 to 65% (v/v), F12 in a final concentration of about 5 to 15% (v/v), M171 in a final concentration of about 15 to 30% (v/v) and FBS in a final concentration of about 1 to 8% (v/v)

14. The method of claim 12 or 13, wherein the culture medium comprises DMEM in a final concentration of about 57.5 to 62.5% (v/v), F12 in a final concentration of about 7.5 to 12.5% (v/v), M171 in a final concentration of about 17.5 to 25.0% (v/v) and FBS in a final concentration of about 1.75 to 3.5% (v/v).

15. The method of any of claims 12-14, wherein the culture medium comprises DMEM in a final concentration of about 61.8% (v/v), F12 in a final concentration of about 11.8% (v/v), M171 in a final concentration of about 23.6% (v/v) and FBS in a final concentration of about 2.5% (v/v).

16. The method of any of claims 12-15, wherein the culture medium further comprises
(i) Epidermal Growth Factor (EGF) in a final concentration of 1 ng/ml to 20 ng/ml, or
(ii) wherein the culture medium comprises EGF in a final concentration of 10 ng/ml, or
(iii) wherein the culture medium comprises Insulin in a final concentration of 1 μg/ml to 10 μg/ml, or
(iv) wherein the culture medium comprises Insulin in a final concentration of 5 μg/ml, or
(v) wherein the culture medium further comprises at least one of the following supplements: adenine, hydrocortisone, and 3,3′,5-Triiodo-L-thyronine sodium salt (T3), or
(vi) wherein the culture medium comprises all three of adenine, hydrocortisone, and 3,3′,5-Triiodo-L-thyronine sodium salt (T3),
wherein optionally the culture medium comprises adenine in a final concentration of 0.01 to 0.1 μg/ml adenine, hydrocortisone in a final concentration of 0.1 to 10 μg/ml hydrocortisone and/or 3,3′,5-Triiodo-L-thyronine sodium salt (T3) in a final concentration of 0.5 to 5 ng/ml.

17. The method of any of claims 12-16, wherein the mesenchymal stem cells of the umbilical cord are human or red deer mesenchymal stem cells of the umbilical cord.

18. A method of producing a conditioned medium,
the method comprising:
(a) cultivating mesenchymal stem cells of the umbilical cord in a culture medium comprising DMEM (Dulbecco's modified eagle medium), F12 (Ham's F12 Medium), M171 (Medium 171) and FBS (Fetal Bovine Serum);
(b) removing the mesenchymal stem cells of the umbilical cord from the culture medium;
(c) optionally cultivating the mesenchymal stem cells of the umbilical cord in a further culture medium, optionally wherein the further cell culture medium comprises a water-soluble antioxidant,
wherein the conditioned medium is obtained by collecting the cell culture medium.

19. The method of claim 18, wherein the further cell culture medium does not comprise a growth factor and/or is a serum-free medium and/or wherein the cells are cultivated at a concentration of about 1 million cells per 1 ml.

20. The method of claim 18 or 19, wherein the mesenchymal stem cells of the umbilical cord are human or red deer mesenchymal stem cells of the umbilical cord.

21. The method of any of claims 18-20, wherein the further medium is a basal medium suitable for cultivation of mesenchymal stem cells, preferably a serum free medium.

22. The method of any of claims 18-21, wherein the further medium is selected from the group consisting of Dulbecco's Modified Eagle Medium (DMEM), DMEM-F12, RPMI media, EpiLIfe medium, and Medium 171.

23. The method of any of claims 18-22, wherein the conditioned medium is diluted in a carrier medium.

24. The method of any of claims 1-17, wherein the conditioned medium is obtained or obtainable by the method according to any of claims 18-23.

25. A conditioned medium derived from mesenchymal stem cells of the umbilical cord obtained or obtainable by the method according to any of claims 18-23.

26. A composition comprising the conditioned medium of claim 25.

27. The composition of claim 26, wherein the composition further comprises an anti-inflammatory compound.

28. The composition of claim 27, wherein the anti-inflammatory compound is selected from the group consisting of curcumin, sulfacetamide, a non-steroidal anti-inflammatory compound, niacinamide (vitamin B3), calendula, sea buckthorn oil, chamomile extract, an extract of Centella asiatica and mixtures thereof.

29. The composition of claim 27 or 28, wherein the composition further comprises a humectant.

30. The composition of clam 29, wherein the humectant is selected from the group consisting of glycerine, propylene glycol, triethylene glycol, tripropylene glycol, sorbitol, hexylene glycol, butylene glycol, urea, collagen and mixtures thereof.

31. The composition of claim 30, wherein the humectant comprises glycerine, propylene glycol or both glycerine and propylene glycol.

32. The composition of any of claims 26 to 31, wherein the composition comprises the conditioned medium derived from cultivation of mesenchymal stem cells of the umbilical cord in an amount of about 5 to 40% (v/v) of the total volume of the composition, the humectant in amount of about 10 to 30% (v/v) of the total volume of the composition, and the anti-inflammatory compound in an amount of about 1.0 to about 3.0% (v/v) of the composition or in an amount of 0.5 to 5.0% (w/w) of the total weight of the composition.

33. The composition of claim 32, wherein the composition comprises the conditioned medium derived from cultivation of mesenchymal stem cells of the umbilical cord in an amount of about 10 to 30% (v/v) of the total volume of the composition, the humectant in amount of about 15 to 25% (v/v) of the total volume of the composition, and the anti-inflammatory compound in an amount of about 1.0 to about 3.0% (v/v) of the composition or in an amount of 0.75 to 3.0% (w/w) of the total weight of the composition.

34. The composition of any of claims 26 to 31, wherein the composition comprises the conditioned medium derived from cultivation of mesenchymal stem cells of the umbilical cord in an amount of about 5 to 40% (w/w) of the total weight of the composition, the humectant in amount of about 10 to 30% (w/w) of the total weight of the composition, and the anti-inflammatory compound in an amount of about 0.5 to 5.0% (w/w) of the total weight of the composition.

35. The composition of any of claims 26 to 34, wherein the composition comprises
conditioned medium derived from cultivation of mesenchymal stem cells of the umbilical cord,
glycerine, propylene glycol or both glycerine and propylene glycol, and
curcumin.

36. The composition of any of claims 26 to 35, wherein the composition further comprises a solvent/carrier.

37. The composition of claim 36, wherein the solvent/carrier is water or an aqueous buffer solution.

38. The composition of any of claims 26 to 37, wherein the composition further comprises a preservative.

39. The composition of claim 38, wherein the preservative is selected from the group consisting of 1,2-hexanediol, benzyl alcohol, benzyl benzoate, benzalkonium chloride, citric acid, a paraben, a formaldehyde-releasing preservative, anisic acid, salicylic acid, sodium salicylate, chlorphenism, and stearalkonium chloride.

40. The composition of any of claims 36 to 39, wherein the composition comprises
water in an amount of about 50 to about 65% (v/v), preferably about 55 to about 58% (v/v), most preferably in an amount of 57% (v/v) of the total volume of the composition
glycerin in an amount of about 10 to about 35% (v/v), preferably about 15 to about 25% (v/v), most preferably about 20% (v/v) of the total amount of the composition
conditioned medium in an amount of about 10 to about 35% (v/v), preferably about 15 to about 25% (v/v), most preferably 20% (v/v) of the total amount of the composition,
1,2-hexanediol in an amount of about 1 to about 55% (v/v), preferably about 1.5 to about 3.5% (v/v), most preferably 2% (v/v) of the total amount of the composition,
curcumin in an amount of about 0.5 to about 5.0% (w/w), preferably of about 0.75 to about 2.5% (w/w), most preferably 1.0% (w/w) of the total weight of the composition.

41. The composition of any of claims 36 to 39, wherein the composition comprises
water in an amount of about 40 to about 65% (w/w), preferably about 45 to about 55% (w/w), most preferably in an amount of about 52% (w/w) of the total weight of the composition,
the conditioned medium in an amount of about the umbilical cord in an amount of about 5 to about 40% (w/w) of the total weight of the composition, preferably in an amount of about 10% (w/w) to about 30% (w/w) or about 12.5 to about 25% (w/w), most preferably in a amount of about 17.5 to 20.0% (w/w) %,
glycerin and/or propylene glycol in amount of about 10 to 30% (w/w) of the total weight of the composition, preferably in an amount of about 10% (w/w) to about 30% (w/w) or about 12.5 to about 25% (w/w), most preferably in an amount of about 17.5 to 20.0% (w/w) of the total weight of the composition, and
curcumin in about 0.5 to 5.0% (w/w) of the total weight of the composition, preferably about 0.75 to about 2.5% (w/w) or 0.8 to about 1.5% (w/w), most preferably about 0.9 to 1.0% (w/w) of the total weight of the composition.

42. The composition of any of claims 36 to 41, wherein the composition further comprises one or more of a gelling agent, a buffering agent and an emollient.

43. The composition of claim 42, wherein the gelling agent is selected from the group consisting of a poly(acrylic acid), pectin, starch, an alginate such as sodium alginate, gelatin, a cellulose derivative and polyvinyl alcohol clay.

44. The composition of claim 42 or 43, wherein the buffering agent is selected from the group consisting of a sodium phosphate salt, calcium carbonate, sodium bicarbonate, citric acid, triethanolamine and a Good's buffer.

45. The composition of claim 44, wherein the Good's buffer is selected from the group consisting of HEPES, Tris, BisTris, Glycylglycine, MOPS (3-(N-morpholino) propanesulfonic acid), and Tricine.

46. The composition of any of claims 42 to 45, wherein the emollient is selected from the group consisting of Olive Oil PEG-7 Esters, triglycerides, lanolin, polyhydric alcohols, and esters of fatty acids (e.g. isopropyl myristate, dioctyl sebecate and dioctyl maleate).

47. Use of conditioned medium derived from cultivation of mesenchymal stem cells of the umbilical cord for the manufacture of a pharmaceutical composition for inducing, stimulating and/or promoting healing of a wound or of a damaged skin area, wherein the use comprises topically administering conditioned medium derived from cultivation of mesenchymal stem cells of the umbilical cord to the skin of a subject surrounding the wound or the damaged skin area.

48. Use of conditioned medium derived from cultivation of mesenchymal stem cells of the umbilical cord for the manufacture of a pharmaceutical composition for preventing formation and/or recurrence of a wound or of a damaged skin area in a subject being at risk of developing a wound or a damaged skin area, wherein the method comprises topically administering to the skin of a subject conditioned medium derived from cultivation of mesenchymal stem cells of the umbilical cord.

49. Use of conditioned medium derived from cultivation of mesenchymal stem cells of the umbilical cord for the manufacture of a pharmaceutical composition for treating rosacea, psoriasis, eczema, dermatitis, topical steroid withdrawal syndrome, epidermolysis bullosa, or skin damage caused by fragile skin, wherein the method comprises topically administering to the skin of a subject conditioned medium derived from cultivation of mesenchymal stem cells of the umbilical cord.

50. The use of any of claims 47 to 49, wherein the conditioned medium derived from cultivation of mesenchymal stem cells of the umbilical cord is obtained or obtainable by the method according to any one of claims 19-24.

51. The use of claim 47, wherein the method does not comprise administering the conditioned medium onto the wound or the damaged skin area.

52. The use of any of claims 47-51, wherein the conditioned medium is administered in the form of a gel, a cream or a liquid.

53. The use of any of claims 47-52, wherein the skin to be treated is the intact skin.

54. The use of any of claims 47-53, wherein the skin to be treated is diabetic skin or thin (weak) skin.

55. The use of any of claims 47-54, wherein the wound or damaged skin area is a chronic wound or a chronically damaged skin.

56. The use of any of claims 47-55, wherein the damaged skin area is a wound.

57. The use of any of claims 47-56, wherein the wound or damaged skin area is selected from the group consisting of diabetic foot ulcer (DFU), pressure ulcer, a wound due to chronic limb ischemia, a wound due to chronic limb-threatening limb ischemia, venous leg ulcer, a surgical wound, a stab wound, a burn wound, and a bit wound.

58. The use of any of the preceding claims 47-57, wherein the conditioned medium is derived from cultivation of a mesenchymal stem cell population of the amniotic membrane of the umbilical cord, from cultivation of a mesenchymal stem population of the Wharton's jelly of the umbilical cord (WJ), from cultivation of a perivascular (PV) mesenchymal stem cell population, or from cultivation of a mixed mesenchymal stem cell population of the umbilical cord (MC).

59. The use of any of the preceding claims 47-58, wherein the conditioned medium is obtained or obtainable by cultivation of mesenchymal stem cells of the umbilical cord in a culture medium comprising DMEM (Dulbecco's modified eagle medium), F12 (Ham's F12 Medium), M171 (Medium 171) and FBS (Fetal Bovine Serum).

60. The use of claim 59, wherein the culture medium comprises DMEM in a final concentration of about 55 to 65% (v/v), F12 in a final concentration of about 5 to 15% (v/v), M171 in a final concentration of about 15 to 30% (v/v) and FBS in a final concentration of about 1 to 8% (v/v)

61. The use of claim 59 or 60, wherein the culture medium comprises DMEM in a final concentration of about 57.5 to 62.5% (v/v), F12 in a final concentration of about 7.5 to 12.5% (v/v), M171 in a final concentration of about 17.5 to 25.0% (v/v) and FBS in a final concentration of about 1.75 to 3.5% (v/v).

62. The use of any of claims 59-61, wherein the culture medium comprises DMEM in a final concentration of about 61.8% (v/v), F12 in a final concentration of about 11.8% (v/v), M171 in a final concentration of about 23.6% (v/v) and FBS in a final concentration of about 2.5% (v/v).

63. The use of any of claims 59-62, wherein the culture medium further comprises
(i) Epidermal Growth Factor (EGF) in a final concentration of 1 ng/ml to 20 ng/ml, or
(ii) wherein the culture medium comprises EGF in a final concentration of 10 ng/ml, or
(iii) wherein the culture medium comprises Insulin in a final concentration of 1 μg/ml to 10 μg/ml, or
(iv) wherein the culture medium comprises Insulin in a final concentration of 5 μg/ml, or
(v) wherein the culture medium further comprises at least one of the following supplements: adenine, hydrocortisone, and 3,3′,5-Triiodo-L-thyronine sodium salt (T3), or
(vi) wherein the culture medium comprises all three of adenine, hydrocortisone, and 3,3′,5-Triiodo-L-thyronine sodium salt (T3),
wherein optionally the culture medium comprises adenine in a final concentration of 0.01 to 0.1 μg/ml adenine, hydrocortisone in a final concentration of 0.1 to 10 μg/ml hydrocortisone and/or 3,3′,5-Triiodo-L-thyronine sodium salt (T3) in a final concentration of 0.5 to 5 ng/ml.

64. The use of any of claims 59-63, wherein the mesenchymal stem cells of the umbilical cord are human or red deer mesenchymal stem cells of the umbilical cord.

65. The use of a composition as defined in any of claims 26-46
i) for inducing, stimulating and/or promoting healing of a wound or of a damaged skin area, wherein the use comprises topically administering conditioned medium derived from cultivation of mesenchymal stem cells of the umbilical cord to the skin of a subject surrounding the wound or the damaged skin area, or
ii) for preventing formation and/or recurrence of a wound or of a damaged skin area in a subject being at risk of developing a wound or a damaged skin area, wherein the method comprises topically administering to the skin of a subject conditioned medium derived from cultivation of mesenchymal stem cells of the umbilical cord, or
iii) for treating rosacea, psoriasis, eczema, dermatitis, topical steroid withdrawal syndrome, epidermolysis bullosa, or skin damage caused by fragile skin, wherein the method comprises topically administering to the skin of a subject conditioned medium derived from cultivation of mesenchymal stem cells of the umbilical cord.

66. The use of claim 65 i), wherein the method does not comprise administering the conditioned medium onto the wound or the damaged skin area.

67. The use of claim 65 or 66, wherein the skin to be treated is the intact skin.

68. The use of any of claims 65-67, wherein the skin to be treated is diabetic skin or thin (weak) skin.

69. The use of any of claims 65-68, wherein the wound or damaged skin area is a chronic wound or a chronically damaged skin.

70. The use of any of claims 65-69, wherein the damaged skin area is a wound.

71. The use of any of claims 65-70, wherein the wound or damaged skin area is selected from the group consisting of diabetic foot ulcer (DFU), pressure ulcer, a wound due to chronic limb ischemia, a wound due to chronic limb-threatening limb ischemia, venous leg ulcer, a surgical wound, a stab wound, a burn wound, and a bit wound.
